(19) United States
(12) Patent Application Publication
(10) Pub. No.: US 2012/0136070 A1
(43) Pub. Date: May 31, 2012

(54) KETAL ESTERS OF ANHYDROPENTITOLS AND USES THEREOF

(75) Inventors: Sergey Selifonov, Plymouth, MN (US); Feng Jing, Snellville, GA (US); Ning Zhou, St. Paul, MN (US)

(73) Assignee: XL TERRA, INC., Plymouth, MN (US)

(21) Appl. No.: 13/345,430

(22) Filed: Jan. 6, 2012

Related U.S. Application Data
(63) Continuation of application No. 13/375,120, filed on Feb. 17, 2012.
(60) Provisional application No. 61/182,161, filed on May 29, 2009.

(51) Int. Cl. A61K 47/34; C08G 69/40; C09D 167/04; C09D 177/00; C09J 167/04; C09D 11/00; C08K 5/109; C08K 5/1545; C08K 5/053; C08G 63/88; C08G 69/46; C11D 3/37; C08G 63/06; C09J 177/00

(52) U.S. Cl. 514/772.7; 528/361; 528/332; 524/599; 524/606; 524/280; 524/110; 524/387; 521/182; 521/183; 521/184; 510/365; 510/476

(57) ABSTRACT

The present disclosure relates to the preparation of ketal compounds from anhydropentitols and oxocarboxylates; derivatives, homopolymers, and copolymers thereof; and various compositions, formulations, and articles derived therefrom.

This application is a continuation of U.S. application Ser. No. 13/375,120, filed Nov. 29, 2011, which is a national stage application under 35 U.S.C. 371 of International Patent Application No. PCT/US2010/036621, filed May 28, 2010, which claims benefit of U.S. Provisional Application No. 61/182,161, filed May 29, 2009; the contents of which are herein incorporated by reference in their entirety.

### FIELD OF THE INVENTION

New chemical compositions based on acetals and ketals of oxocarboxylate esters with the cyclic ethers of pentitols are disclosed as well as polymeric compositions formed from them.

### BACKGROUND

International Patent Publication No. WO 2009/032905 and U.S. Patent Publication No. 2008/0242721 disclose the reaction products of triols, such as glycerol, 1,1,1-trimethylolpropane, or 1,1,1-trimethylolethane, with esters of various oxocarboxylates including alkyl levulinates, alkyl acetoacetates, and alkyl pyruvates:

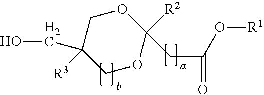

wherein a is 0 or an integer between 1 and 12, b is 0 or 1, R₁ is any substituent, R₂ is hydrogen or an alkyl group, and R₃ is hydrogen, methyl or ethyl. These compounds all feature one free hydroxyl group and one carboxylate ester, acid, or salt per molecule; thus, the compounds may be referred to as “hydroxyketal esters”. The hydroxyl moiety and the ester moiety of the hydroxyketal esters are available for further reactions, including self-condensation to form a homopolyester.

In some embodiments, hydroxyketal esters have the advantage of employing raw materials that have their basis in renewable bio-based feedstocks, wherein “renewable” is used as defined in ASTM D6866. The hydroxyketal ester formed from glycerol, a triol, and a levulinate ester is one such example:

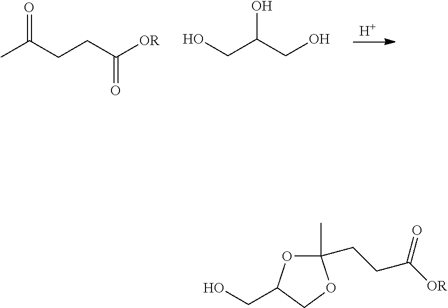

Glycerol is a byproduct of biodiesel fuel synthesis. Levulinic acid and levulinate esters have their basis in renewable plant-based feedstocks. Such starting materials are advantageously used to replace petroleum based feedstocks, such as those used to make the phthalates and many other commercially useful polymers.

However, the glass transition temperature of some species of homopolymers based on the hydroxyketal esters is below 25° C. For example, the homopolymer of the hydroxyketal ester formed from glycerol and a levulinate ester is about 5° C.-10° C., depending on variables such as molecular weight. Further, many such polymers have very little crystalline content. Some such polymers are rubbery materials at typical room temperature, and uses of such polymers are limited to applications such as adhesives and the like. A polymer must have a glass transition temperature above ambient use temperature in order for it to be useful as a rigid, structural or load-bearing article, such as an automobile part, a table top, a utensil or container for holding food or beverages, and the like. “Ambient” temperature, for many applications, ranges between 15° C. and 45° C., and is often higher than 25° C. In some cases, ambient temperature is as high as 100° C., for example in applications where contact with boiling or near-boiling water is anticipated.

Copolymerization of hydroxyketal esters with other monomers can be employed to raise the overall glass transition temperature of the polymer, impart crystallinity, or both. For example, copolymerization of a hydroxyketal ester with a diol and a terephthalate ester is known to result in a glass transition temperature of the copolymer that is significantly higher than that achievable with the corresponding hydroxyketal ester homopolymer. However, employing phthalates and other petroleum-based monomers results in the reduction of renewable bio-based feedstock content in the resulting polyester.

It is desirable to employ 100% renewable bio-based feedstocks to form useful monomeric compounds. It is desirable to use such monomeric compounds to form homopolyesters and other polymeric materials having glass transition temperatures that are higher than ambient use temperature. It is particularly desirable to form such materials having glass transition temperatures of 100° C. or greater. It is desirable to form such materials that can be subjected to temperatures of up to 100° C., or even greater than 100° C., without softening or loss of strength or integrity. It is desirable to provide polymers that are transparent to visible light for many applications.

### SUMMARY OF THE INVENTION

We have found a new class of hydroxyester monomers that feature a bicyclic structure. The monomers are formed by acetalization or ketalization of oxocarboxylates with the cyclic ethers of certain pentitols. The monomers have one hydroxyl functionality and one ester functionality per molecule. The acetal or ketal moieties of the monomers are cyclic and constitute one ring of the bicyclic fused ring structure, in conjunction with the cyclic ether moieties. The pentitols and oxocarboxylates that are useful to make the monomers are, in some embodiments, based on 100% renewable bio-based feedstocks.

The present invention further includes methods employed to make the new compounds. The present invention further includes useful reaction products of the new monomeric species that are useful as e.g. plasticizers, tackifiers, coalescing solvents, and the like in various formulations.

The present invention further includes dimers, oligomers and polymers based on the new compounds. The oligomers and polymers include products of both homopolymerization and copolymerization. One or more homopolymers of the invention unexpectedly possess very high glass transition temperatures, in some embodiments greater than 100° C.

The present invention further includes compositions and formulations incorporating these new dimers, oligomers, and polymers.

The present invention further includes articles made from the dimers, oligomers, or polymers of the invention, or from formulations that include the dimers, oligomers, polymers, plasticizers, tackifiers, coalescing solvents, and the like of the invention.

Additional advantages and novel features of the invention will be set forth in part in the description that follows, and in part will become apparent upon examination of the following, or may be learned through routine experimentation upon practice of the invention.

### BRIEF DESCRIPTION OF THE DRAWINGS

FIG. 1 shows the gas chromatograph portion of a GC-MS spectrum of a compound of the invention (Total Ion Current).

FIG. 2 shows the mass spectrum portion of an electron-ionization mass spectrum of a compound of the invention.

FIG. 3 shows the mass spectrum portion of an electron-ionization mass spectrum of a compound of the invention.

FIG. 4 shows a ¹H NMR spectrum of a compound of the invention.

FIG. 5 shows the gas chromatograph portion of a GC-MS spectrum of a compound of the invention (Total Ion Current).

FIG. 6 shows a ¹H NMR spectrum of a compound of the invention.

FIG. 7 shows a DSC scan for a compound of the invention.

FIG. 8 shows a gel permeation chromatogram for a compound of the invention.

FIG. 9 shows a ¹H NMR spectrum of a compound of the invention.

FIG. 10 shows a ¹H NMR spectrum of a compound of the invention.

FIG. 11 shows a ¹H NMR spectrum of a compound of the invention.

### DETAILED DESCRIPTION OF THE INVENTION

In embodiments, the compounds of the invention have the general formula I

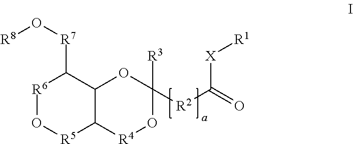

wherein;
a is 0 or an integer of 1 to 12;
X is O or NR, wherein R is hydrogen or a linear or branched alkyl group having between 1 and 6 carbons;
R¹ is hydrogen, a metal cation, an organic cation, a linear, branched, or cyclic alkyl, a linear, branched, or cyclic alkenyl, alkynyl, aryl, alkaryl, or a polymeric moiety;
each R² is independently methylene, alkylmethylene, or dialkylmethylene;
R³ is hydrogen, an alkynyl group, or a linear, branched, or cyclic alkyl or alkenyl group having 1 to 18 carbon atoms, or an aryl or alkaryl group;
one of R⁴ and R⁵ is a methylene, alkylmethylene, or dialkylmethylene group and the other is a covalent bond; and
one of R⁶ and R⁷ is a methylene, alkylmethylene, or dialkylmethylene group and the other is covalent bonds, with the proviso that R⁵ and R⁶ are not simultaneously a covalent bond, and
R⁸ is hydrogen or an acyl group having a linear, branched, or cyclic alkyl or alkenyl group, aryl group, or aralkyl group.

As used herein, “polymeric moiety” means a residue of a polymerized hydroxylated compound. The polymeric moiety is not particularly limited as to structure or molecular weight; the only limitation is that the polymer from which the polymeric moiety is derived has at least one hydroxyl or amino group capable of forming a carboxylate ester or carboxamide linkage when reacted with a carboxylic acid, carboxylate ester, carboxamide, or carboxylate salt. In some embodiments, residues of polyols such as polyester polyols, polyether polyols, and the like constitute the structure of the polymeric moiety.

In embodiments, one or more of R¹, R², R³, R⁷, and R⁸ further have one or more heteroatoms, including, for example, oxygen, nitrogen, sulfur, halogen, silicon, or phosphorus. Compounds I are referred to herein in some instances as compound I carboxylates. The compound I carboxylates include, as used herein, carboxylic acid, carboxylate ester, carboxylate salt, or carboxamide moieties. Compounds I are synthesized from the cyclic ethers of pentitols (pentols), which in turn are synthesized by known dehydration processes. For example, the conversion of xylitol, a pentitol, to a racemic mixture of 1,4-anhydroxylitol (“1,4-AX”) isomers called “xylitan” is known:

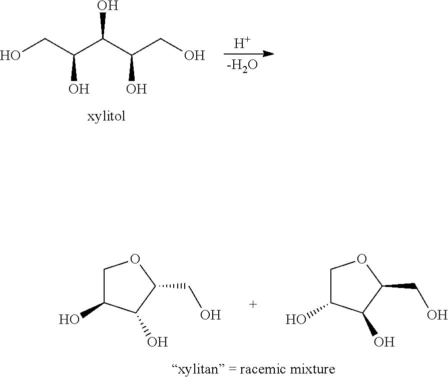

Xylitol is one of four isomers of 1,2,3,4,5-pentapentanol. Other isomers include ribitol and arabitol. Xylitol is a renewable bio-based sugar alcohol that can be extracted from the fibers of many plants as well as some trees, including various berries, corn husks, oats, birch trees, and mushrooms. More practically, xylitol is industrially obtained by a known process of hydrogenation of xylose. The latter can be produced by known hydrolysis techniques allowing for utilization of a range of non-food pentosane-containing biomass sources such as corn cobs, corn stover, cereal straw, cane bagasse, wood residues, paper pulp process liquors and the like.

Dehydration reactions to yield cyclic structures are also carried out, in embodiments, with various stereoisomers of xylitol as well as other pentitols. Also formed, in some dehydration reactions, are 1,5-anhydropentitol (“1,5-AP”) adducts having three adjacent hydroxyl moieties attached to the six-membered ring. Collectively, the anhydropentitol (“AP”) stereoisomers may be generally represented by the structures

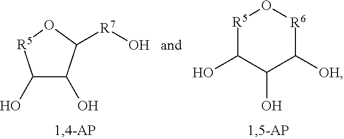

where R⁵, R⁶, and R⁷ are as defined for compound I above, with the proviso that none of R⁵, R⁶, and R⁷ in the 1,4-AP and 1,5-AP structures are covalent bonds. Typically, the dehydration reaction to form the ether ring is brought about by heating the pentitol in the presence of a strong mineral acid, such as sulfuric acid, with concomitant removal of water. Representative examples of such reactions are described in e.g. Kurzewska et al., J. Carbohydrate Chem. 2004, 23(4), 169-77 and Carson et al., J. Am. Chem. Soc. 1945, 67(10), 1808-10. In embodiments, the reaction conditions described in Carson et al. are particularly advantageous for selectively preparing 1,4-anhydroxylitol. Further to the teaching of Carson et al., in some embodiments the reaction is carried out with greatly reduced amounts of strong mineral acids to yield 1,4-anhydroxylitol of high purity and low color. For example, in some embodiments about 50-200 ppm of acid catalyst is employed; in other embodiments about 10-100 ppm of acid catalyst is employed. In some embodiments the reaction is carried out at about 160° C.-180° C. In some embodiments the reaction is carried out by heating for about 1-3 hours. In embodiments, the reaction results in isolated molar yields of 77%-85%, in some embodiments more than 85% molar yield.

In some embodiments, the compounds I of the invention are formed the by reaction of one molar equivalent of an oxocarboxylate with one molar equivalent of an AP to form a cyclic ketal. Oxocarboxylates are compounds having one carboxylate group and one ketone or aldehyde group, which is represented herein as

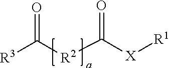

wherein R¹, R², R³, X, and a are as defined for compound I. The ketone or aldehyde group of the oxocarboxylate can form a cyclic ketal or acetal in the presence of compounds having two hydroxyl moieties disposed either on contiguous carbons, or on two carbons having one carbon disposed between them. Such reactions are disclosed, for example, in Patent Publication Nos. WO 2007/062118, WO 2009/032905, and WO 2009/048874 as well as references cited therein. Any of the methods disclosed therein that are employed to make acetals and ketals from oxocarboxylates are, in various embodiments, also employed to make compounds I of the invention from cyclic ethers and oxocarboxylates.

On 1,4-APs, a pair of hydroxyl moieties are disposed on contiguous carbon atoms and a pair of hydroxyl moieties are disposed on carbon atoms having one carbon atom spaced between them. Cyclic ketal formation may occur at either site, leaving one unreacted hydroxyl group; reaction with the hydroxyls bonded to contiguous carbon atoms results in a five-membered cyclic ketal ring, and reaction with hydroxyls bonded to carbon atoms having one carbon atom between them results in a six-membered cyclic ketal ring. Cyclic ketal formation with a 1,5-AP results in a 5-membered ketal ring, provided that at least one pair of hydroxyl groups in 1,5-AP is in a relative cis orientation. These three isomeric possibilities are represented below. Notably, more than one of these isomers are present, in some embodiments, depending on thermodynamic, kinetic, and stereoisomeric factors affecting ring formation of both the cyclic ether and the cyclic ketal.

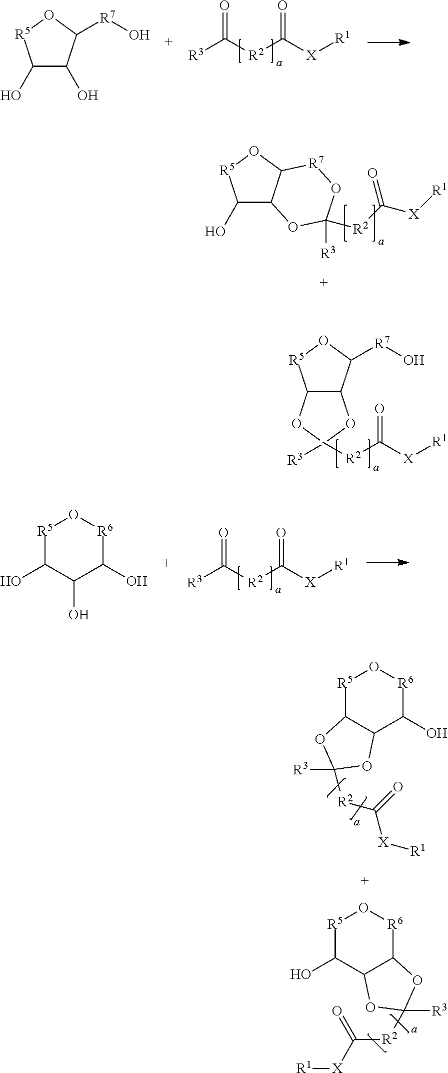

In some embodiments of the reaction to form compounds I, the stereochemistry of the AP affects selectivity in the reaction with the oxocarboxylate. For example, in the xylitol conversion to xylitan example set forth above, each 1,4-AX racemate contains a pair of hydroxyl moieties disposed on contiguous carbon atoms, and a pair of hydroxyl moieties disposed on carbon atoms having one carbon atom spaced between them. However, the two contiguous hydroxyls are disposed in a trans configuration relative to one another. This configuration favors ketalization at the exocyclic hydroxymethyl site, that is, ketalization of the hydroxyls disposed on carbon atoms having one carbon atom spaced between them. Thus, the major product of the reaction of xylitan with an oxocarboxylate is a six-member cyclic ketal ring. The entirety of the favored reaction is shown below:

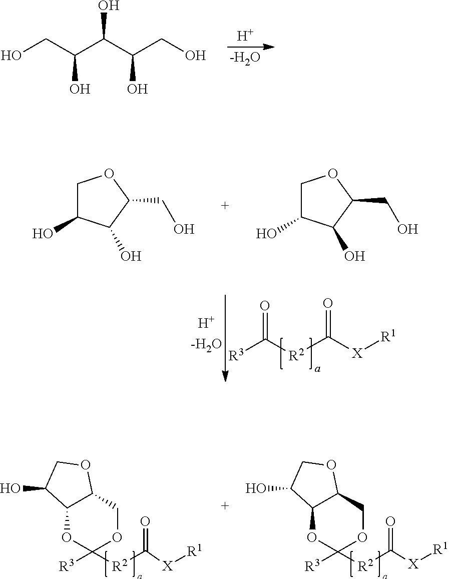

wherein a, X, R¹, R², and R³ are as defined for compound I.

Compounds useful in forming compounds I of the invention include various anhydropentitols (AP), and oxocarboxylates. The particular species of pentitol employed to form the AP is not particularly limited, but proximity of hydroxyls must be such that dehydration to form a 5- or 6-membered cyclic ether is possible, leaving at least two hydroxyls disposed on either contiguous carbon atoms or on carbon atoms having one carbon disposed between them. Useful pentitols include, without limitation as to various possible stereoisomers, linear or branched pentitols. Linear or branched pentitols include, for example, pentane-1,2,3,4,5-pentol, hexane-1,2,3,4,5-pentol, 6-chlorohexane-1,2,3,4,5-pentol, 6-bromohexane-1,2,3,4,5-pentol, 6-(octylamino)hexane-1,2,3,4,5-pentol, 5-(1-hydroxyethylamino)hexane-1,2,3,4,6-pentol, 5-aminohexane-1,2,3,4,6-pentol, 1-deoxy-1-(methylamino)-D-glucitol, 2-deoxy-2-fluoro-D-glucitol, 6-ethoxy-6-ethylsulfanylhexane-1,2,3,4,5-pentol, and the like.

Oxocarboxylates include keto acids, keto esters, semialdehydes, and semialdehyde esters. “Keto acid” refers to an oxocarboxylate having at least one ketone moiety and one carboxylic acid moiety. A keto acid may have more than one ketone functionality or more than one carboxylic acid functionality. The keto acid is not particularly limited as to additional moieties or functionalities present in addition to the ketone and carboxylic acid functionalities. In some embodiments, the keto acid may also contain one or more heteroatoms. Some examples of suitable keto acids include pyruvic acid, acetoacetic acid, levulinic acid, 5-aminolevulinic acid, oxaloacetic acid, α-ketobutyric acid, α-ketoglutaric acid, α-ketoisovaleric acid, 5-ketohexanoic acid, α-ketoisocaproic acid, α-ketoadipic acid, 3-ketoadipic acid, 2-keto-4-methylthiobutyric acid, 4-acetylbutyric acid, 2-keto-3-bromobutyric acid, phenylpyruvic acid, 2-keto-3-phenylpropanoic acid, 2-ketopentanoic acid, 3-ketohexanoic acid, 4-ketohexanoic acid, 2-ketooctanoic acid, 3-ketooctanoic acid, 4-ketooctanoic acid, 7-ketooctanoic acid, 2-keto-4-pentenoic acid, 13-keto-9,11-octadecadienoic acid, 4-ketostearic acid, 9-ketopalmitic acid, 4-ketoheptanedioic acid, penicillic acid, 8-keto-8-aminopelargonic acid, 2-keto-5-aminovaleric acid, 2-succinylamino-6-oxoheptanedioic acid, 2-oxo-3-butynoate, 3-keto-6-acetamidohexanoate, and the like. Additionally, a keto acid may contain hydroxyl or mercapto functionality provided it is protected, e.g. by one or more trimethylsilyl or t-butyl groups, or one or more other protecting groups known in the art.

In some embodiments of the invention, the keto acid employed is levulinic acid (4-oxopentanoic acid). Levulinic acid is an abundant feedstock that is can be prepared on an industrial scale by acidic degradation of hexoses and hexose-containing polysaccharides such as cellulose, starch, sucrose, and the like, or more efficiently, by acid-catalyzed rearrangement of a non-food renewable starting material furfuryl alcohol in the presence of water. In other embodiments, pyruvic acid, and acetoacetic acid are other acids are employed.

“Keto ester” refers to the carboxylic ester derivative of the one or more keto acid compounds. The ester group is, in embodiments, the reaction product of a keto acid and an alkanol, wherein the alkanol contains at least one hydroxyl and one organic group that is a linear, branched, or cyclic alkyl or alkenyl group having 1 to 18 carbon atoms, or an aryl or alkaryl group, wherein the alkyl, alkenyl, aryl, or alkaryl groups optionally have one or more additional functional groups that can include, for example, halogen, ester, amine, thiol, ether, or silane functionalities, and are not particularly limited. Thus, in embodiments, the organic group is methyl or ethyl; a linear or branched isomer of an alkyl group such as propyl, butyl, pentyl, hexyl, septyl, octyl, nonyl, decyl, undecyl, dodecyl, tetradecyl, cetyl, or stearyl; a cycloalkyl group such as cyclohexyl, cyclooctyl, norbornyl, and the like; an alkynyl group such as ethynyl, 3-methylpent-1-yn-3-yl, tetradec-9-yn-1-yl, and the like; an aryl and alkaryl group such as phenyl, benzyl, tolyl, xylyl, 5-phenylpent-1-yl, and the like. The alkyl, alkenyl, alkynyl, aryl, or alkaryl may primary, secondary or tertiary, and may additionally have one or more functional groups; thus, for example, the organic group is, in some embodiments, 1,1,1-trichloro-2-methyl-2-propyl, 5-fluoro-1-pentyl, 5-amino-1-pentyl, 5-benzyloxy-1-pentyl, 5-methoxy-1-pentyl, 3-nitro-2-pentyl, 4-methylthio-1-butyl, 1-carboxyhex-6-yl, propionamid-2-yl, and the like. In embodiments, the organic group is a protecting group, for example trimethylsilyl, phosphonooxy, or a phosphatidyl group. The composition of the organic group is not particularly limited.

In some embodiments of the invention, esters of levulinic acid, pyruvic acid, or acetoacetic acid are employed as the keto esters in the polyols. For example, ethyl levulinate or n-butyl levulinate can be employed in some embodiments of the invention. Levulinic esters are based on levulinic acid, an abundant feedstock that is prepared on an industrial scale by acidic degradation of hexoses and hexose-containing polysaccharides such as cellulose, starch, sucrose, and the like. More efficiently, levulinic esters are produced by a known acid-catalyzed rearragement of a non-food renewable starting material, furfuryl alcohol, in the presence of corresponding alcohol.

“Keto amide” refers to the carboxamide derivative of the one or more keto acid compounds. The carboxamide group is, in embodiments, the reaction product of a keto acid and a primary or secondary amine, wherein the amine has the structure HNRR′, wherein the R group is as defined for compound I and R′ is an organic group that is generally the same as the organic group described for the keto ester compounds above.

“Semialdehyde” refers to an oxocarboxylate having at least one aldehyde functionality and one carboxylic acid functionality. A compound may have more than one aldehyde functionality or more than one carboxylic acid functionality. The semialdehyde is not particularly limited as to additional moieties or functionalities present in addition to the aldehyde and carboxylic acid functionalities. In some embodiments, the semialdehyde may also contain one or more halogen, ester, phosphate, amine, thiol, ether, or silane groups. Some examples of suitable semialdehydes include 2-oxoethanoic acid (glyoxylic acid), 3-oxopropanoic acid (malonic semialdehyde), 4-oxobutanoic acid, 5-oxopentanoic acid, 6-oxohexanoic acid, 7-oxoheptanoic acid, α-formylglycine, aspartic semialdehyde, 3-oxo-2-(phosphonooxy)-propanoic acid (tartronic semialdehyde wherein the hydroxyl group is protected by phosphate), 2-methyl-3-oxopropanoic acid (methylmalonic semialdehyde), succinic semialdehyde, adipic Semialdehyde, 5-glutamyl semialdehyde, allysine, 2-aminomuconic semialdehyde, 4-amino-5-oxopentanoic acid, N-acetylglutamic semialdehyde, 2-amino-3-(3-oxoprop-1-enyl)-but-2-enedioic acid, and N2-succinyl-L-glutamic-5-semialdehyde. Many other semialdehydes are available by carrying out ozonolysis of unsaturated fatty acid esters to form an aldehyde moiety at an unsaturated site, as described by Criegee, Angew. Chem. Int. Ed., 1975, 87, 745. The aldehyde moiety of semialdehyde can be also present in the form of a semiacetal or an acetal.

“Semialdehyde ester” refers to an ester derivative of one or more carboxylate functionalities of any of the above described semialdehyde compounds. The nature of the ester groups are generally the same as those described above for the keto ester compounds. “Semialdehyde amide” refers to a carboxamide derivative of one or more carboxylate functionalities of any of the above described semialdehyde compounds. The nature of the carboxamide groups are, in embodiments, the same as those described above for the keto amide compounds.

Methods useful in making compounds I include known methodologies for making both the APs and their ketals with oxocarboxylates. As disclosed above, the dehydration of pentitols is a known reaction. As such, any of the techniques described in the literature for dehydration of polyols with concomitant cyclic ether formation are suitably employed to form the cyclic ethers of pentitols. Such methods include generally mild reaction conditions. In embodiments, a strong protic acid is employed as a catalyst for the reaction. However, the method is not particularly limited as to the particular species of acid catalyst employed. Strong protic acids (Brønsted-Lowry acids) are those that have a Kₐ of 55 or greater. Examples of suitable strong protic acid catalysts include sulfuric acid, arylsulfonic acids and hydrates thereof, such as p-toluenesulfonic acid monohydrate, perchloric acid, hydrobromic acid, and hydrochloric acid. Generally, the amount of acid catalyst employed in the dehydration of linear pentitols is about 50 to about 10,000 ppm based on the weight of starting pentitol. In some embodiments, the catalyst is incorporated into, or onto, or covalently bound to, a solid support material. Resin beads, membranes, porous carbon particles, zeolite materials, and other solid support materials may be functionalized with acid moieties that are, in embodiments, covalently bound or strongly sorbed to one or more surfaces of the solid support. In a nonlimiting example, sulfonated resin is used in embodiments of the invention, which provide active sulfonic acid groups that are covalently bonded to the resin.

In embodiments, the reaction is carried out by heating the pentitol in the presence of the catalyst, employing conditions whereby water is removed from the reaction vessel. Such conditions include heating the pentitol and catalyst to above 100° C. and allowing water to distill; employing vacuum to assist in removal of water, in embodiments at temperatures below 100° C.; fractional distillation; employing molecular sieves, superabsorbent materials, or another means of removing water within the reaction vessel itself; employing an inert solvent that forms an azeotrope with water and distilling the azeotrope; selective membrane filtration; dialysis; or any other technique known in the art for drying materials.

In some embodiments, the AP is isolated and/or purified prior to reaction with an oxocarboxylate. Isolation or purification is accomplished, for example, by distillation, membrane separation, column separation, or any other standard separation technique familiar in the art In other embodiments, the second step of the reaction to form compounds I is carried out in the same reaction vessel that is employed to form the cyclic ether. The AP formation and isolation is accomplished, in various embodiments, in batch or continuous reaction processes, using one of devices known in the art, such as batch or continuous feed distillation columns, wiped film evaporators, spinning film evaporators, rotary evaporators, falling film evaporators and other similar equipment.

In some embodiments, reaction conditions employed to form compounds I from the direct reaction of AP and oxocarboxylate are the same as those disclosed in International Patent Publication No. WO 09/048,874, the content of which is incorporated herein by reference in its entirety; and US Patent Publication No. 2008/0242721, the contents of which is incorporated herein by reference in its entirety. The reaction conditions disclosed in the incorporated applications are usefully employed to form compounds I including the racemic mixture or cis/trans isomners thereof from oxocarboxylates and APs having free hydroxyl groups available for ketalization. These methods incorporate strong acids as catalysts and, optionally, excess molar complement of oxocarboxylate. Thus, in embodiments, an oxocarboxylate is simply added to the reaction vessel containing the AP, and compounds I formed therein. The reaction to form compounds I is carried out, in embodiments, in a single pot using batchwise reaction conditions. In other embodiments, the reaction is carried out in a continuous reaction by addition of oxocarboxylate in a downstream location along a reaction pathway, after dehydration of pentitol has been accomplished upstream. In still other embodiments, the reaction is carried out in two separate steps, with our without purification of the reaction intermediates and products between the two steps.

In some embodiments, the second step of the reaction to form compounds I is carried out employing transketalization. Transketalization methods employed to form ketals from oxocarboxylates and acyclic triols such as glycerol are described in International Patent Application No. WO 2009/146202, the contents of which are incorporated herein by reference in their entirety. Other methods of transketalization are known and may usefully be employed in conjunction with the current invention. For example, transketalization of pentaerythritol is disclosed by Lukes, J. Org. Chem., 1961, 26 (7), pp 2515-2518. Pryde, U.S. Pat. Nos. 3,183,215 and 3,223,683 discloses ketal exchange of the dimethoxy ketal of azelaic semialdehyde with pentaerythritol. These and any other known methods of transketalization are usefully employed with one or more embodiments of the current invention.

In some embodiments employing transketalization to reach the compounds I of the invention, transketalization is a process wherein an intermediate ketonide is formed by the reaction of the AP with a ketone; then the ketonide is reacted with an oxocarboxylate to yield the compound I and the ketone by an exchange reaction. A representative example of ketonide formation is the reaction of 1,4-anhydroxylitol (xylitan) with methyl isobutyl ketone (MIBK), wherein two stereoisomers of xylitan form two cis and two trans stereoisomers of the corresponding ketonide with the asymmetric ketone:

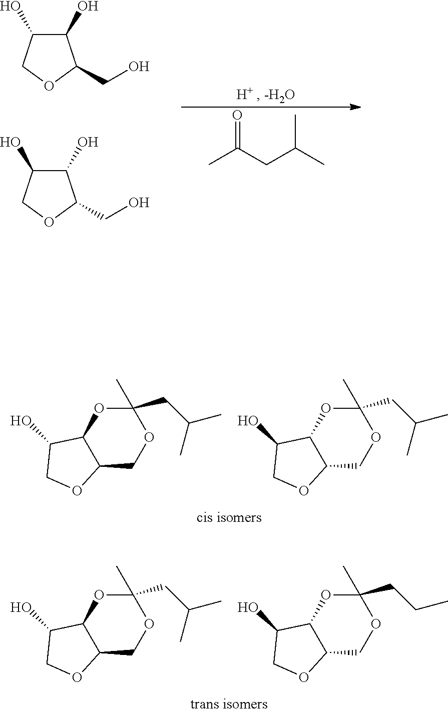

whereas symmetrical ketones, for example acetone or diethyl ketone, result in two stereoisomers corresponding to the two xylitan isomers. The cis and trans configurations denote the relationship of the two ketonide alkyl groups to the hydroxyl and hydroxymethyl groups of the anhydroxylitol molecules.

The ketonide intermediate is then subjected to transketalization, an exchange reaction between the ketonide and the oxocarboxylate, to yield compound I and the original ketone. For example, employing the embodiment shown above, transketalization of the MIBK ketonide of xylitan with ethyl levulinate (ethyl 4-oxopentanoate) results in the formation of four possible isomers of the corresponding anhydroxylitol levulinate ketal:

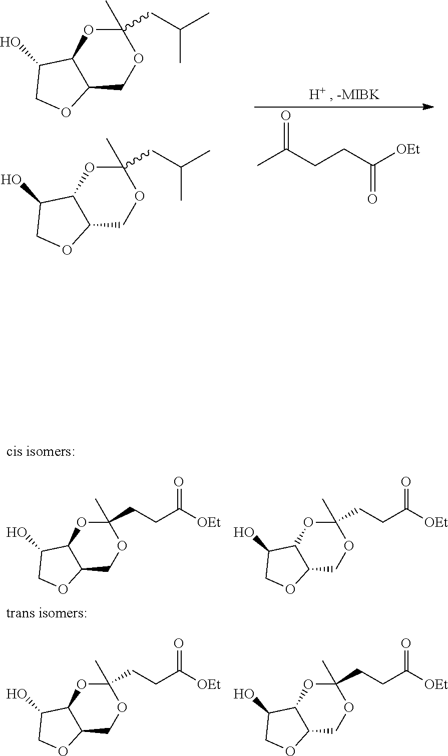

wherein the cis and trans labels are applied in similar fashion to the MIBK ketonides described above.

In some embodiments, transketalization is advantageous in that water is removed in the formation of the ketonide intermediate to provide a dry ketal compound for reaction to form compound I. This in turn allows for greater purity of the resulting final product, often obviating purification steps such as distillation, because side reactions involving water are obviated. Obviating distillation is advantageous from an industrial standpoint; additionally, in some embodiments, it is advantageous to avoid the high temperatures required in some embodiments to accomplish the distillation of compound I. In some embodiments, transketalization is advantageous in that low temperatures can be employed, for example as low as about 20° C., in other embodiments between 0° C. and the reflux temperature of the ketone, in still other embodiments between 0° C. and about 200° C., to carry out the transketalization exchange reaction. The ability to use mild reaction conditions is advantageous in many industrial processes. In some embodiments, transketalization provides a means of recycling the ketone employed in forming the intermediate ketonide. Recycling of the ketone is of particular importance where the ketonide formation/transketalization of the AP, or the overall reaction from pentitol to compound I, or both are carried out using continuous or semi-continuous processes.

In still other embodiments, transketalization of 1,4-anhydroxylitol is advantageous because the kinetics of the transketalization exchange of the 1,4-anhydroxylitol ketonide leads to the predominant formation of trans isomers; that is, greater than 1:1 ratio of trans:cis isomers in the 1,4-anhydroxylitol levulinate ketal. In some embodiments the transketalization reaction of the 1,4-anhydroxylitol ketonide with the oxocarboxylate occurs in such a fashion that the trans isomeric products are strongly favored. For example, in some embodiments, between 1:1 and 500:1 ratio of cis:trans isomers are formed in the product mixture when transketalization of a 1,4-anhydroxylitol ketonide is employed to synthesize compound I; in other embodiments between 2:1 and 300:1 ratio of cis:trans isomers; in other embodiments between 3:1 and 100:1 ratio of cis:trans isomers; in still other embodiments between 5:1 and 10:1 ratio of cis:trans isomers are formed in the reaction of a 1,4-anhydroxylitol ketonide to form the corresponding ketal carboxylate (compound I). Without wishing to be limited by theory, we believe the trans isomers are favored in certain embodiments due to lower steric crowding caused, for example in the reaction shown above, by the alkyl ester (ethyl propanoat-yl) moiety disposed in the trans formation vs. the cis formation relative to the disposition of the bicyclic ring structure.

In some embodiments, trans isomers of ketal carboxylates of 1,4-anhydroxylitol form a crystalline phase at common ambient temperatures, for example between 18° C.-23° C. In some such embodiments, a neat mixture of cis and trans isomers will, upon standing, form a crystalline-appearing solid phase that, when isolated, optionally washed, and analyzed, is found to be composed of a very high ratio of trans:cis isomers of compound I. In some embodiments, the solids thus formed are essentially 100% trans isomers of a ketal carboxylate of 1,4-anhydroxylitol, wherein measurable amounts of cis isomers are attributable to the solid being wetted with cis isomers from the mother liquor of the neat mixture. In other embodiments, the solids are measured to have trans:cis ratios of about 500:1 to 5:1, or about 300:1 to 10:1, or about 100:1 to 25:1. In some embodiments, trans ketal carboxylates of 1,4-anhydroxylitol form a solid, crystalline-appearing phase at temperatures below common ambient temperatures; for example, by using known recrystallization techniques such as dissolving a mixture of isomers of a ketal carboxylate of 1,4-anhydroxylitol in a solvent, in some embodiments by warming the mixture of isomers and solvent, and lowering the temperature of the solution formed until a precipitate is observed. As with the neat mixtures, the solids that form, when isolated, are measured to have trans:cis ratios of about 500:1 to 5:1, or about 300:1 to 10:1, or about 100:1 to 25:1.

Thus, in various embodiments of compounds I that are not limited to just the formation of 1,4-anhydroxylitol levulinate ketals, enrichment of a trans isomer or isomers is accomplished. Enrichment is accomplished in various embodiments, for example, by recrystallization, including melt or solution recrystallization using static or falling film crystallization techniques known in the art, for example, for purification of lactide, or triglycerides. In other embodiments, enrichment is accomplished by carrying out ketalization or transketalization reactions favoring formation of high ratios of trans:cis in the compound I formed, or in other embodiments by other means such as column separation of product isomers and the like.

In some embodiments, such as in ketalization or transketalization with alkyl levulinate, reaction conditions favor a selective crystallization of a portion of anhydroxylitol levulinate ketal having a high trans-isomer content (e.g. trans:cis isomer ratio in excess of about 20), while the non-crystallized liquid portion of the reaction mixture is maintained under protic acid-catalyzed trans-cis equilibrium conditions. This technique permits stereoselective synthesis of the trans-isomer.

Ketones that are useful in forming the intermediate ketonides of AP are linear, branched, or cyclic dialkyl ketones, optionally having one or more double bonds. Examples of useful dialkyl ketones include propan-2-one (acetone), butan-2-one (methyl ethyl ketone, or MEK), 3-methylbutan-2-one, 3,3-dimethylbutan-2-one, pentan-2-one, pentan-3-one (diethyl ketone, or DEK), 2-methylpentan-3-one, 2,4-dimethylpentan-3-one, 2,2-dimethylpentan-3-one, 2,2,4-trimethylpentan-3-one, 2,2,4,4-tetramethylpentan-3-one, 3-methylpentan-2-one, 4-methylpentan-2-one (methyl isobutyl ketone, or MIBK), 4,4-dimethylpentan-2-one, hexan-2-one, hexan-3-one, 5-methylhexan-2-one, 5-methylhexan-3-one, 2-methylhexan-3-one, 4-methylhexan-3-one, 2,2-dimethylhexan-3-one, 2,5-dimethylhexan-3-one, 2,2,5,5-tetramethylhexan-3-one, heptan-2-one, heptan-3-one, heptan-4-one, 5-methylheptan-3-one (ethyl amyl ketone), 6-methylheptan-3-one, 2-methylheptan-4-one, 2,6-dimethylheptan-4-one, octan-2-one, octan-3-one, octan-4-one, 2-methyloctan-3-one, nonan-2-one, nonan-3-one, nonan-4-one, nonan-5-one, 2-methylnonan-3-one, 2,6,8-trimethylnonan-4-one, decan-2-one, decan-3-one, decan-4-one, decan-5-one, undecan-2-one, undecan-3-one, undecan-4-one, undecan-5-one, undecan-6-one, 2-methylundecan-4-one, dodecan-2-one, dodecan-3-one, dodecan-4-one, hexadecane-10-one, and the like. Dialkyl ketones optionally contain one or more halogen atoms; thus, for example, 1-fluoropropan-2-one, 1,3-dichloropropan-2-one, 1-bromo-3,3-dimethylbutan-2-one, and 5-chloropentan-2-one are also useful ketones for forming the polyketal precursors of the invention. In some embodiments, asymmetric ketones are employed in the formation of the ketonides. Asymmetric ketones are those having two different alkyl groups attached to the oxo carbon. Examples of asymmetric ketones include MEK and MIBK.

In some embodiments where transketalization is employed, it is important to select a ketone that has a higher volatility than the oxocarboxylate at the temperature selected for the transketalization reaction. By selecting the oxocarboxylate and ketone to provide this relative volatility, the transketalization is, in embodiments, driven to completion by stripping off the ketone as the ketonide is transketalized with the oxocarboxylate. In embodiments, addition of heat, or application of vacuum, or both is employed to accomplish the stripping of the ketone as it forms. In some embodiments, the same general reaction conditions are employed for the ketalization of the AP with the ketone and the transketalization of the ketonide with the oxocarboxylate. In some such embodiments, ketonide formation and transketalization are carried out in subsequent steps in a single reaction vessel by simply adding the ketone to the AP, optionally heating the mixture, and removing water as it forms; then adding the oxocarboxylate and allowing it to react with the ketonide while removing the ketone as it forms. In embodiments, heat, vacuum, or both are employed along with an acid catalyst similarly to the acid catalysis described above.

In an alternative embodiment of the transketalization approach, a ketal oxocarboxylate is formed as an intermediate, then the ketal carboxylate is reacted with an AP in a transketalization to yield two moles of an alkanol per mole of oxocarboxylate, and the product compound I. Thus, in some embodiments the dimethoxy or diethoxy, or other dialkoxy adducts of oxocarboxylates are employed in a transketalization reaction with an AP to yield two moles of methanol or ethanol per mole of compound I. Ketal or acetal adducts based on an oxocarboxylate and a diol are, in some embodiments, also suitably employed in a transketalization reaction with an AP. In one example, methyl 3,3-dimethoxypropionate, which is the dimethoxy adduct of methyl formylacetate or methyl 3-oxopropionate, is suitably employed in a transketalization reaction with 1,4-anhydroxylitol to yield the corresponding anhydroxylitol acetoacetate acetal. This embodiment is represented below:

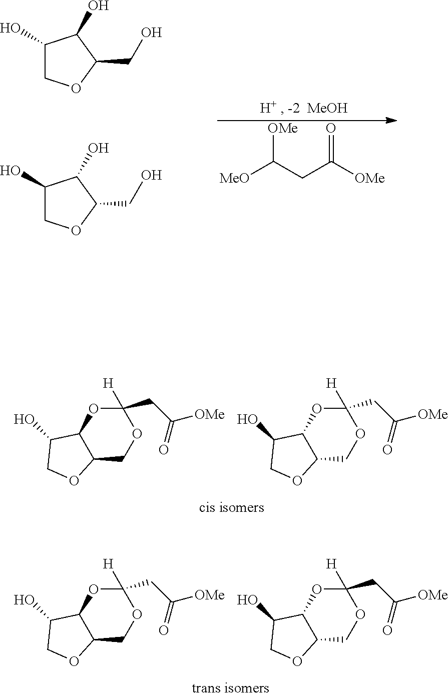

As is described above, it is preferred that the alkanol or diol product of the transketalization have higher volatility than the AP, so that the transketalization is driven to completion, in embodiments, by the removal of the product alkanol or diol as the reaction proceeds.

The process to form compounds I is carried out, in various embodiments, in a batch operation, in a continuous operation, or in a semi-continuous operation. The reagents and acid catalyst are, in embodiments, mixed during the reaction by employing any of a variety of techniques known in the art. For example, mechanical mixing by a propeller, impeller, or a mechanical agitator such as a shaker, roller, or tumbler can be used. Passive mixing, such as by a static mixer, may also be employed. In some embodiments, the reagents are mixed in a reactor with active or passive mixing, optionally including some quantity of the product ketal or acetal to aid in miscibility. In some embodiments, the reaction mixture is heated and a vacuum optionally applied to remove substantially all water formed in the reaction. In some embodiments, the water is removed by distillation; in other embodiments water is removed by distillation of its azeotrope with the oxocarboxylate; in still other embodiments, the water is removed by including molecular sieves, superabsorbent materials, or another means of removing water within the reaction vessel itself. In some embodiments, the resulting product mixture containing compound I also contains excess oxocarboxylate as well as the acid catalyst. In such embodiments, the product mixture is further subjected to a distillation to remove excess oxocarboxylate, and further, in embodiments, to distill out a majority of the product compound I. The distillation can be carried out in a batch process or in a continuous fashion, using one of devices known in the art, such as batch or continuous feed distillation columns, wiped film evaporators, spinning film evaporators, rotary evaporators, falling film evaporators and other similar equipment. In embodiments, any oxocarboxylate, AP, or acid catalyst remaining in the reaction vessel is subsequently re-used by mixing with additional fresh reagents.

In some embodiments, the compounds I of the invention include certain cyclic species, or compound I lactones, conforming to generalized structure shown below:

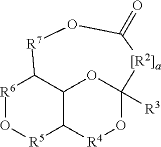

wherein R², R³, R⁴, R⁵, R⁶, R⁷, and a are as defined for compound I. The compound I lactones are formed by intramolecular condensation of the hydroxyl functionality of R⁷ with the carboxylic acid or ester functionality of the molecule. In embodiments, the compound I lactones are a side product of one or more reactions to form compound I or in one or more reactions of compound I to form a homopolyester or copolyester, as is described below. In still other embodiments, reaction conditions are optimized to maximize the yield of the compound I lactone. In some embodiments, compound I lactone is the major product recovered upon depolymerization of a compound I homopolymer; in other embodiments, it is a side product formed in a depolymerization reaction of a compound I homopolymer. The compound I polymers are described in greater detail below.

In some embodiments, the stereochemistry of compound I prevents the formation of cyclic species by placing the carboxyl group out of proximity of the hydroxyl group. For example, the reaction of xylitol to form xylitan places the hydroxyl and the carboxylic groups in a trans relationship relative to one another. In some embodiments, when xylitan is functionalized with an oxocarboxylate, the corresponding compound I lactone will not form due to the relative placement of the carboxylic group and the hydroxyl group.

The compounds I, including compound I lactones, are useful in a number of applications. For example, in some embodiments where R¹ of compound I is an alkyl group and X is O, compounds I are useful as plasticizers or coalescing solvents in one or more polymeric formulations. In some such applications, it is advantageous to employ R¹ that has more than 6 carbon atoms. In some such embodiments, it is useful to incorporate an R¹ group having more than 6 carbons by transesterification of an ester group of compound I wherein R¹ is an alkyl group having 6 carbons or less. By employing transesterification, very long carbon chains, such as C₈-C₃₆ and higher, are easily imparted to compound I either before or after formation of the compound I carboxylate. Standard transesterification techniques are suitably employed to facilitate the transesterification.

In some embodiments of compound I carboxylates where X is O and R¹ is a cation, for example sodium, ammonium, and the like, compounds I are surfactants in one or more formulations. Such ionic versions of compounds I are easily formed using standard saponification techniques in conjunction with either compound I or the oxocarboxylate starting compounds. Further, the hydroxyl moiety of compound I is available for a transesterification reaction, e.g. with an alkyl ester, for example an alkyl octanoate, decanoate, hexadecanoate, and the like, to provide a hydrophobic “tail” for surfactant applications. In such embodiments, R⁸ of compound I is a carboxy group, for example acetate, propanoate, pentanoate, and the like including carboxy groups having between 1 and 36 carbon atoms. Linear, branched, or cyclic hydrocarbon tails are applicable, in various embodiments, for one or more surfactant applications. Fatty acid esters, preferably renewably sourced fatty acid esters, are also available for transesterification to functionalize one or more compounds I for surfactant type applications. In forming a hydrophobic tail, the number of carbon atoms in the hydrocarbon tail is not particularly limited. In various embodiments, the hydrocarbon tail has between 1 and 36 carbon atoms, or between 6 and 18 carbon atoms, or between 8 and 16 carbon atoms. The hydrocarbon tail further includes, in some embodiments, one or more heteroatoms. In some such embodiments, the heteroatoms are O, N, halogen such as Cl or F, S, Si, or P.

In some embodiments of compound I, R⁸ has a structure corresponding to the residue of a ketal ester:

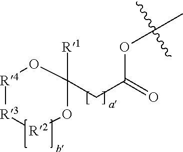

wherein
a′ is 0 or an integer of 1 to 12;
b′ is 0 or 1, such that b=0 indicates a 5-membered ring and b=1 indicates a 6-membered ring;
R′¹ is hydrogen or methyl; and
R′², R′³, and R′⁴ are independently methylene, alkylmethylene, or dialkylmethylene.

In such embodiments, the ketal esters are generally the reaction products of oxocarboxylates and diols having hydroxyl groups disposed in a 1, 2 or 1,3 configuration. Such compounds are known in the literature. The reaction products of compounds I with ketal esters are referred to as compound I ketal esters. The reaction products include, in some embodiments, compound I diols, compound I bis-diols, compound I aminoalcohols, and polyesters II and various adducts thereof as described below, wherein one or two terminal moieties corresponding to the R⁸ group of compound I are ketal ester endgroups; or wherein additional reaction products of ketal esters with copolymerized compounds to form copolyesters II are provided. Compound I ketal esters are usefully employed in one or more formulations as plasticizers, solvents, adjuvants, surfactants, or additives when one or more polymers, colorants, surfactants, solvents, other materials, or a combination thereof are further included; wherein the one or more polymers, surfactants, plasticizers, or solvents include in some embodiments one or more compounds of the invention.

In some embodiments of compound I, R¹ has a structure corresponding to the residue of glycerol carbonate:

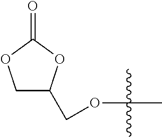

wherein the corresponding alcohol is the reaction product of glycerol and a dialkyl carbonate or phosgene, made for example by the processes described in Okutsu et al., U.S. Pat. No. 6,495,703 or any of the references cited therein. The reaction products of compounds I with glycerol carbonate are referred to as compound I glyceryl carbonates. The reaction products of glycerol carbonate include, in some embodiments, polyesters II and various adducts thereof as described below, wherein one or two terminal moieties corresponding to the R¹ group of compound I are glyceryl carbonate endgroups; or wherein additional reaction products of the glycerol carbonate with copolymerized compounds to form copolyesters II are provided. Compound I glyceryl carbonates are usefully employed in one or more formulations as plasticizers, solvents, adjuvants, surfactants, or additives when one or more polymers, colorants, surfactants, solvents, other materials, or a combination thereof are further included; wherein the one or more polymers, surfactants, plasticizers, or solvents include in some embodiments one or more compounds of the invention.

In some embodiments of compound I, X is NH or NR. In such embodiments, compounds I are referred to as compound I amides. Compound I amides are useful as additives, for example as surfactants, in a number of formulations. Compound I amides are synthesized, in embodiments, from compound I carboxylate esters using standard reaction methods employed in the literature to form alkyl amides from alkylamines and esters. In some embodiments, the reaction is carried out using a catalyst. In some such embodiments, the catalyst 1,5,7-triazabicyclo[4.4.0]dec-5-ene is suitably employed as a catalyst that provides for the amidation to take place using mild conditions and resulting in high conversions of ester to amide moieties.

Primary and secondary amines are employed, in embodiments, in reactions to form compound I amides. Nonlimiting examples of suitable amines include any of those having one or two linear, branched, or cyclic alkyl groups, or aromatic, or aralkyl groups having between 1 and 36 carbon atoms, or between 2 and 18 carbon atoms, or between 2 and 8 carbon atoms. Suitable amines further include, in some embodiments, one or more heteroatoms. In some such embodiments, the heteroatoms are O, N, S, Si, P, or a halogen such as Cl, Br, or F.

In some embodiments, compounds I are reacted with an aminoalcohol to form one or more compounds I having an amide group and, where R⁸ is hydrogen, two hydroxyls; thus, in embodiments, such compounds are referred to as compound I diols. In such embodiments, X is NR and R¹ contains one hydroxyl moiety. The compound I diols are synthesized using any method available in the literature for reacting an amine with an ester to form an amide linkage. In some embodiments, the reaction is carried out using a catalyst. In some such embodiments, the catalyst 1,5,7-triazabicyclo[4.4.0]dec-5-ene, or a titanium tetraalkoxide, is suitably employed as a catalyst that provides for the amidation to take place using mild conditions and resulting in high conversions of ester to amide moieties. Nonlimiting examples of suitable aminoalcohols that form compound I diols of the invention when reacted with a compound I carboxylate include 2-aminoethanol, 3-aminopropan-1-ol, isopropanolamine, 2-aminopropan-1-ol, 2-aminobutan-1-ol, 2-amino-3-methylbutan-1-ol, 2-amino-4-methylpentan-1-ol, 6-aminohexan-1-ol, 1-amino-3-chloropropan-2-ol, 7-aminobicyclo[2.2.2]octan-8-ol, 2-aminopyridin-3-ol, 2-amino-4-phenylphenol, 5-aminonaphthalen-1-ol, 4-(4-aminophenyl)phenol.

In other embodiments, compounds I are reacted with a diamine to form one or more compounds I having, in embodiments wherein R⁸ is hydrogen, one hydroxyl and one amine, referred to as a compound I aminoalcohol. In such embodiments, compound I has X═NR and R¹ further comprises a primary or secondary amino group. In some embodiments where compound I is reacted with a diamine, two moles of compound I react with one mole of diamine to form a compound I bis-diol. This structure of a compound I bis-diol is represented below:

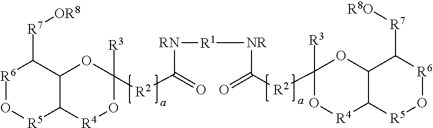

wherein R¹-R⁸ and a are as defined for compound I. In some embodiments of the structure shown above, R⁸ is hydrogen. It will be appreciated that depending on stoichiometry and reaction conditions employed when a diamine is reacted with compound I carboxylate, either a compound I bis-diol or a compound I aminoalcohol, or a mixture thereof, will form. The compound I aminoalcohols and compound I bis-diols are synthesized from compound I carboxylates using any method available in the literature for reacting an amine with an ester or an amide (e.g. transamidation) to form an amide linkage. In some embodiments, the reaction is carried out using a catalyst. In some such embodiments, the catalyst 1,5,7-triazabicyclo[4.4.0]dec-5-ene, or a titanium tetraalkoxide, or a mixture or combination thereof, is suitably employed in conjunction with mild conditions to result in high conversions of ester to amide moieties. Nonlimiting examples of suitable diamines that form compound I aminoalcohols or compound I bis-diols of the invention when reacted with a compound I carboxylate include hydrazine, ethane-1,2-diamine, 1,6-hexanediamine, but-2-ene-1,4-diamine, Metformin, butane-1,4-diamine, propane-1,2-diamine, piperazine, 2,2,4-trimethyl-1,6-hexanediamine, 2,4,4-trimethyl-1,6-hexanediamine, benzene-1,3-diamine, 2-methylbenzene-1,3-diamine, 4-chlorobenzene-1,3-diamine, methanediamine, and the like.

The compound I diols, compound I aminoalcohols, and compound I bis-diols are useful, in embodiments, as crosslinkers, tackifiers, solvents, or surfactants in one or more formulations further including one or more polymers, colorants, surfactants, solvents, or a combination thereof; wherein the one or more polymers, surfactants, plasticizers, or solvents include in some embodiments one or more additional compounds of the invention. In other embodiments, the compound I diols, compound I aminoalcohols, and compound I bis-diols are usefully reacted with a ketal ester, the structure of which is defined above, to form ketal ester adducts of any of the hydroxyl or amino moieties of the compound I diols, compound I aminoalcohols, and compound I bis-diols. Such ketal ester adducts are usefully employed in various embodiments as plasticizers, tackifiers, surfactants, or cosolvents in one or more formulations when one or more polymers, colorants, surfactants, solvents, or a combination thereof are further included; wherein the one or more polymers, surfactants, plasticizers, or solvents include in some embodiments one or more additional compounds of the invention.

In still other embodiments, the dual functionality of the compound I diols, bis-diols, and aminoalcohols wherein R⁸ is hydrogen make them useful in polymerization reactions to form polymers based on diols or aminoalcohols. Such polymers include polyesters, poly(amide esters), polyurethanes, poly(urethane urea)s, polycarbonates, poly(amide carbonate)s, acrylate and methacrylate adducts and polymerized products thereof, epoxidized adducts and polymerized products thereof, allyl adducts and polymerized products thereof, and copolymers of these as well as blends thereof with other polymers or compounds, including polymers and additional compounds of the invention, for example as plasticizers, solvents, surfactants, tackifiers, crosslinkers, and the like. In embodiments, the reactions, reagents, catalysts, solvents, and methods used to form the polyesters, polyamide esters, polyurethanes, polyurethane ureas, polycarbonates, polyamide carbonates, acrylate and methacrylate adducts and polymerized products thereof, epoxidized adducts and polymerized products thereof, allyl adducts and polymerized products thereof, and copolymers of these employ methods in the literature familiar to those of skill in the art of polymer synthesis. Such polymers have varying content of renewable bio-based feedstocks, wherein “renewable” is used as defined in ASTM D6866 wherein renewable content is provided by the compounds of the invention, other monomers employed, or a combination thereof. In some embodiments, content of renewable feedstocks in such polymers ranges from about 1% to 100% by weight of the polymeric compound, or from about 20% to 100%, or about 50% to 100%, or about 80% to 100% by weight of the polymeric compound formed using one or more compound I diols, bis-diols, and aminoalcohols.

In embodiments, compound I diols, compound I aminoalcohols, compound I bis-diols, or a combination thereof wherein all R⁸ are hydrogen are used as reactants for thermoset systems in which they react with crosslinking resins such as polyisocyanates, blocked polyisocyanates, polyfunctional epoxides or a methylolated-alkylated amino crosslinker made from one of the following base aminoplasts: urea-formaldehyde, melamine formaldehyde, glycoluril-formaldehyde and benzoguanadine-formaldedhyde. In some such embodiments, compound I diols, compound I aminoalcohols, compound I bis-diols, or a combination thereof are blended with other hydroxyl functional resins selected from the class consisting of acrylic, polyester, alkyd, polyether, epoxy ester and polyurethane resins and used in thermoset systems with the crosslinking resins described above.

Compound I carboxylates having R⁸=hydrogen and X=oxygen contain both hydroxyl functionality and ester or free acid functionality. In some such embodiments, compounds I are polymerized by self-condensation, employing esterification or transesterification reactions, to form a homopolymer. In other such embodiments, compounds I are copolymerized with one or more diols and diacids/diesters and/or other hydroxyesters by similar mechanisms of condensation. In such embodiments, a polymer having at least one repeat unit corresponding to structure II is formed:

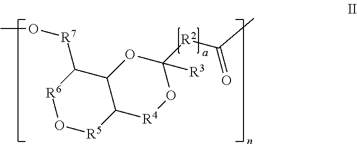

wherein R²-R⁷ and a are as defined for compound I, and n is an integer of between 1 and about 500. Homopolyesters and copolyesters of compound I are referred to herein collectively as “polyesters II.” Polyesters II include homopolyesters II and copolyesters II. In some embodiments, polyesters II have terminal endgroups corresponding to OR¹ and R⁸ of compound I. Such terminal endgroups include any moieties, functional groups, or polymeric groups described above for R¹ or R⁸ of compound I including for example, R⁸=hydrogen, carboxy group, or ketal ester or R¹=hydrogen, alkyl group, or glycerol carbonate. Homopolyesters II having very low values of n, for example n=1-3, are useful as plasticizers, solvents, tackifiers, surfactants in one or more formulations

The homopolyesters II having values of n of 3 or more are characterized by unexpectedly high glass transition temperatures (T_{g}). In embodiments, the homopolyesters have T_{g} values of about 50° C. to 150° C.; in some embodiments between about 75° C. and 130° C.; in still other embodiments between about 100° C. and 125° C. Thus, a high T_{g} polymer is achievable, in embodiments, by employing 100% renewable raw materials such as xylitol or a stereoisomer thereof and levulinate or pyruvate esters. The high T_{g} observed for polyesters II means that they are potentially useful in one or more applications wherein a polycarbonate, polyimide, or certain polyesters such as poly(ethylene terephthalate) (PET) are commonly employed as a rigid, load-bearing materials.

In some embodiments, certain stereoisomers of compounds I lead to differences in observed T_{g} of the corresponding polyesters II. For example, as discussed above, it is possible to isolate or enrich the amount of trans isomers of compounds I based on 1,4-anhydroxylitol and levulinate esters. Enrichment of the trans isomer is accomplished, for example, by recrystallization, ketalization or transketalization reactions favoring formation of high ratios of trans:cis in the 1,4-anhydroxylitol levulinate ketal, or by other means such as column separation of product isomers and the like. In some such embodiments, polyesterification of the 1,4-anhydroxylitol levulinate ketal wherein the ratio of trans:cis is about 3:1 results in a homopolyester II having T_{g} about 105° C.; in some other embodiments, polyesterification of the 1,4-anhydroxylitol levulinate ketal wherein the ratio of trans:cis is about 25:1 results in a homopolyester II having T_{g} about 115° C. From the standpoint of making a homopolyester II with good heat deflection properties in the range of temperatures near the boiling point of water, in embodiments at atmospheric pressure, such differences in Tg between homopolyesters II made with mixtures of monomer I of different stereoisomer compositions is advantageous. For example, a packaging material made from a polyester II can be prepared to have heat deflection sufficient to withstand heating and steam formed during preparation or pre-heating of various food articles in a microwave oven, or to withstand heat of beverages poured into a container comprising such polymer.

In some embodiments, the homopolyesters II of the 1,4-anhydroxylitol levulinate ketal are further characterized by good transparency in the visible spectrum. The combined properties of high T_{g} and good transparency make the homopolyesters II, and their corresponding copolyesters II, suitable for a range of applications.

Copolyesters II include one or more comonomers. Comonomers include dihydric and polyhydric alcohols, diacids or diesters, and hydroxyacids, hydroxyesters, or lactones thereof. Nonlimiting examples of dihydric alcohols useful in forming copolyesters II of the invention include, in embodiments, 1,2-ethanediol (ethylene glycol), 1,2-propanediol (propylene glycol), 1,3-propanediol, 2,2-dimethyl-1,3-propanediol (neopentyl glycol), 2-butyl-2-ethyl-1,3-propanediol, 3-mercaptopropane-1,2-diol (thioglycerol), dithiothreitol, 1,2-butanediol, 1,3-butanediol, 1,4-butanediol, 2,3-butanediol, 2,2,4,4-tetramethyl-1,3-cyclobutanediol, 1,5-pentanediol, 3-methyl-1,5-pentanediol, 1,6-hexanediol, 2-ethyl-1,3-hexanediol, cyclohexane-1,2-diol, cyclohexane-1,4-diol, 1,4-dimethylolcyclohexane, 1,4-dioxane-2,3-diol, 3-butene-1,2-diol, 4-butenediol, 2,3-dibromobutene-1,4-diol, 1,8-octanediol, 1,10-decanediol, 1,12-dodecanediol, benzene-1,2-diol (catechol), 3-chlorocatechol, indane-1,2-diol, tartaric acid, and 2,3-dihydroxyisovaleric acid, diethylene glycol (DEG), triethylene glycol, tetraethylene glycol, dipropylene glycol, tripropylene glycol, tetrapropylene glycol, pentaerythritol, trimethylolpropane, sorbitol, xylitol, anhydroxylitol, erythritol, xylene glycol, 1,3-benzenediol (resorcinol), 1,4-benzenediol (hydroquinone), o, m, or p-benzene dimethanol, o, m, or p-glycol phthalates, o, m, or p-bis-1,2-ethylene glycol phthalates, o, m, or p-bis-1,2-propylene glycol phthalates, o, m, or p-bis-1,3-propylene glycol phthalates, diols prepared by hydrogenation of dimer fatty acids, hydrogenated bisphenol A, hydrogenated bisphenol F, propoxylated bisphenol A, isosorbide, 2-butyne-1,4-diol, 3-hexyne-3,5-diol (SURFYNOL® 82, available from Air Products of Allentown, Pa.) and other alkyne-based polyol products marketed under the SURFYNOL® brand name by Air Products of Allentown, Pa., and polymeric polyols such as polyether polyols based on ethylene glycol, for example CARBOWAX® polyethylene glycols (available from The Dow® Chemical Company of Midland, Mich.), polyether diols and polyols based on propylene glycol or combinations of ethylene glycol and propylene glycol, such as those sold by the The Dow® Chemical Company of Midland, Mich., and polyether glycols such as those produced by the INVISTA™ Company of Wichita, Kans. under the trade name TERETHANE®; polycarbonatediols of varying molecular weights, such as L467m, L600m, and L565m, available from Asahi Kasei Corporation (Tokyo, Japan); polyols based on hydroxylated vegetable oils, such as those sold under the trade name BiOH®, available from the Cargill Company of Wayzata, Minn.; hydroxyl-terminated polybutadienes, such as HTPB R45M, sold by Aerocon Systems of San Jose, Calif., polyols produced by the Everchem Company of Media, Pa., or the Maskimi Polyol Sdn. Bhd. of Kajang, Selango Darul Ehsan, Malaysia, and the polyols employed in the Union Carbide Company (South Charleston, W. Va.) publication by Carey, M. A. et al., “Rapid Method for Measuring the Hydroxyl Content of Polyurethane Polyols” (published on the internet at http://www.polyurethane.org/s_api/doc_paper.asp?CID=1044&DID=4060).

Nonlimiting examples of diacids and triacids or esters thereof, useful in forming copolymers II of the invention include, in embodiments, oxalic acid, malonic acid, succinic acid, adipic acid, pimellic acid, suberic acid, sebacic acid, or a o-, m- or p-phthalic acid, citric acid, benzene 1,2,4-tricarboxylic acid, or any of the other known diacids. Diesters include monohydric alcohol esters of any of the above mentioned diacids or triacids, diesters of carbonic acid (e.g. dialkyl carbonates or cyclic carbonates of glycols); preferably, the monohydric alcohol has between 1 and 6 carbon atoms, but the number of carbon atoms is not particularly limited. In embodiments, copolyesters II are obtained by employing urea or urea compounds, for example carbonyl bislactamates such as carbonyl bis-N-caprolactamate, in one or more reactions to form copolyesters II. Carbonyl bis-N-caprolactamate is well known to react with hydroxyl groups under elevated temperature in a reaction vessel, or in an extruder, and is particularly useful to obtain copolyesters II of increased molecular weight, for example, polymers having Mₙ over about 30,000. In some such embodiments, copolyesters II with improved mechanical properties are obtained.

Nonlimiting examples of hydroxyacids useful in forming copolymers II of the invention include, in embodiments, lactic acid and its cyclic dimer lactide, 2-hydroxybutanoic acid, 3-hydroxypropanoic acid, 4-hydroxybutanoic acid, 2-hydroxy-4-methylsulfanylbutanoic acid, 5-hydroxypentanoic acid, 2-hydroxy-4-methylpentanoic acid, 3-hydroxytetradecanoic acid, 12-hydroxydodecanoic acid, mandelic acid, 2-hydroxybenzoic acid, 3-(4-hydroxyphenyl)prop-2-enoic acid, and the like, and the monohydric alcohol esters of any of the above mentioned hydroxyacids; preferably, the monohydric alcohol has between 1 and 6 carbon atoms, but the number of carbons is not particularly limited.

In some embodiments, a hydroxyketal acid, or ester thereof, is employed as a comonomer in a polymerization to form a copolyester II. Hydroxyketal esters are represented by the structure

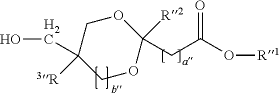

wherein a″ is 0 or an integer between 1 and 12, b″ is 0 or 1, and R″¹, R″₂, and R″₃ are independently hydrogen or alkyl. Such compounds are disclosed in U.S. Patent Publication No. 2008/0242721, the contents of which are incorporated herein in its entirety. Of the hydroxyketal esters, preferred structures are those formed from glycerol and a levulinate ester, for example ethyl levulinate or butyl levulinate. The T_{g} of the homopolymer of the glycerol levulinate ketal is about 5°-10° C., depending on molecular weight. When the glycerol levulinate ketal, ethyl ester, is copolymerized with a compound I, the range of T_{g} available is thus about 5° C. in ranges near 0 mole % of compound I, to about 150° C. at ranges near 100 mole % of compound I.

Polyesters II are not limited in particular by the method employed to make them. In general, any method of polyesterification employed in the literature is suitably employed using esters or free acids of compounds I to form the polyesters II of the invention. In some embodiments, self-condensation or co-condensation of the compound I esters or free acids is carried out in the presence of a catalyst. While the choice of catalyst is not particularly limited within the scope of the invention, a preferred set of embodiments employs an organometallic catalyst, for example a catalyst based on titanium or tin, such as titanium tetrabutoxide (Ti(OBu)₄), or tin (II) octanoate, or organic zirconates. Other suitable catalysts are, for example, organic titanates and zirconates marketed under Tyzor® brand by DuPont deNemours and Co. of Wilmington, Del. In another set of preferred embodiments, catalysts such as tin tetrachloride (SnCl₄) or titanium tetrachloride (TiCl₄) are also suitably employed; however, in such embodiments it is preferred to employ an acid scavenger, such as a tetraalkylammonium hydroxide, in conjunction with the catalyst in order to scavenge any hydrochloric acid that is formed during the reaction. Generally, known techniques of polyesterification involves temperatures in excess of 100° C. and further includes a means to remove the water or monohydric alcohol R¹OH (referring to compound I) that is formed during the reaction. Such techniques also employ some means of efficient mixing and stirring the polymer as the molecular weight builds, because high viscosities are encountered in the final stages of the polymerization.

In some embodiments where R⁸ of compound I or the corresponding endgroup R⁸ of polyester II is hydrogen, the R⁸ groups are employed in the ring opening reaction of one or more lactones to form the corresponding copolyester II. Ring opening polymerization of lactones is carried out using one or more catalysts in conjunction with reaction conditions suitable for ring opening polymerization. Catalysts and reaction conditions employed in such reactions are any of those used in the art for ring opening reactions of lactones. For example, some ring opening polymerization catalysts are based on transition metals such as zinc, tin, or titanium. Without limiting the species of catalysts or reaction conditions employed, any of the catalysts or reaction conditions described in Hori et al., U.S. Pat. No. 5,516,883 or Schechtman et al., U.S. Pat. No. 5,648,452 are useful. Activated carbon as employed by Endo et al., EP1857484 or organic catalysts employed as described in a web-published article from IBM Company of Armonk, N.Y., at www.almaden.ibm.com/st/chemistry/ps/catalysts/RingOpening/ may be used to affect the ring opening polymerization of lactones using the hydroxyl functionalities of the compounds I or polyesters II of the invention as the initiating hydroxyl functionality. The above examples are not limiting as to the type of catalyst or set of reaction conditions that can be employed in a ring opening polymerization of lactones.

Suitable lactones for the ring opening polymerization initiated by one or more polyketal polyols of the invention include, without limitation, propiolactone, pivalolactone, diketene, dimethyldiketene, β-butyrolactone, 4-butyrolactone, 4-valerolactone, ε-caprolactone, 5-ethenyl-5-methyloxolan-2-one, gluconolactone, glucuronolactone, D-galactonolactone, coumarin, hydrocoumarin, ascorbic acid lactone, α-angelicalactone, 2-acetylbutyrolactone, 6-propyloxan-2-one, 6-ethyloxan-2-one, ribonolactone, arabonolactone, λ-nonalactone, bicyclononalactone, 5-nonalactone, λ-decalactone, pantolactone, 2-dehydropantolactone, 5-butoxolan-2-one, isocrotonolactone, 6-hexyloxan-2-one 5-heptyloxolan-2-one, 5-propyloxolan-2-one, 6-[(E)-pent-2-enyl]oxan-2-one, cocolactone, isocitric lactone, 2-hydroxy-6-methylpyran-4-one, 1-oxacyclododecan-2-one, ε-dodecalactone, 1-oxacyclopentadecan-2-one, 1-oxacycloheptadecan-2-one, L-arabino-1,4-lactone, 4-hydroxy-4-methyloxan-2-one, lactide, homoserine lactone, 4-methyl-7-propan-2-yloxepan-2-one, and the like.

In one embodiment of a lactone ring opening polymerization, one or more moieties R⁸═H of compound I or polyester II are employed in the ring opening polymerization of SEGETOLIDE™ (available from Segetis, Inc. of Golden Valley, Minn.) or its dimer to form the corresponding repeat unit based on glycerol levulinate ketal, a hydroxyketal ester moiety as described above. The structure of SEGETOLIDE™ and its dimer, as well as methods for the ring opening polymerization of both compounds, are found in U.S. Patent Publication No. 2008/0242721, the contents of which are incorporated by reference herein in their entirety. The methods disclosed therein are suitable, in embodiments, for initiating the ring opening polymerization using hydroxyl groups of the compounds I of the invention as initiators to give copolyesters II.

In some embodiments, a desirable feature of the polyesters II of the invention is recyclability via depolymerization. In embodiments, thermal depolymerization of polyesters II, including those that have been incorporated into a crosslinked network, employ catalysts. Basic catalysts, for example alkali and alkali-earth alkoxides, hydroxides, or carbonates; trisodium or tripotassium phosphate; disodium or dipotassium hydrogen phosphate, are useful for cleaving polyesters II at the ester linkage. Protic acid catalysts, for example sulfuric acid, hydrochloric acid, toluenesulfonic acid, and phosphoric acid are useful, in embodiments, for catalyzing cleavage of both ester and ketal linkages to form free oxocarboxylates and cyclic ether polyols. Lewis acid type catalysts, for example titanium (IV) catalysts, are employed in embodiments along with one or more additional dihydric alcohols to catalyze cleavage of the ester linkages while leaving the ketal linkages intact.

In some embodiments, the polyesters II of the invention include certain bishydroxy adducts conforming to the structures shown below:

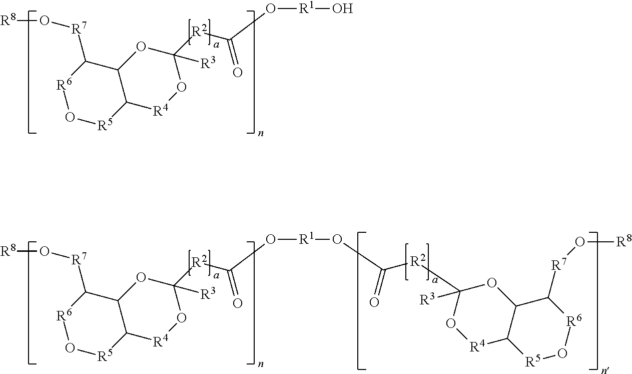

wherein R¹-R⁸, and a are as defined for polyesters II, and n and n′ are between about 1 and 10. These structures correspond to the reaction products of one or more compound I with dihydric alcohols (diols), such as any of those listed herein. Such compounds are referred to generally as polyester II diol adducts. Polyester II diol adducts are useful for developing one or more formulations suitable for replacing present nonrenewable petrochemical-based polymers for thermoplastics, coatings, elastomers, adhesives, sealants and other industry applications. In embodiments where R⁸ of the polyester II diol adducts are hydrogen, the polyester II diol adducts are polyester II diols analogous to the compound I diols and compound I bis-diols. As such, in various embodiments the polyester II diols are useful in the same formulations, and are polymerizable or crosslinkable in the same manner and using the same compounds and methodologies as described above for the compound I diols and compound I bis-diols.

In embodiments, polyester II diol adducts are synthesized from the corresponding compounds I by esterification or transesterification employing standard techniques known in the art, conjunction with one or more diols. Stoichiometry and choice of catalyst, if any, is adjusted to control the degree of self-condensation of compound I, which is competitive with reaction with the diol, and obtain the desired molecular weight and number of repeat units attributable to compound I. The polyester II diol adduct structures are not particularly limited by the methods employed to make them. Adjustment of reaction conditions and the stoichiometric ratio of compound I with dihydric alcohol is suitably adjusted to result in the desired polyester II diol adduct structure, as will be appreciated by those of skill.

It will be appreciated that polyester II triol adducts, polyester II tetrol adducts, and polyester II adducts of polyhydric alcohols of higher functionality that are otherwise related structurally to the polyester II diol adducts are also formed, in some embodiments, by partial or complete functionalization of polyhydric alcohols with one or more compounds I. Thus, triols such as glycerol, 1,1,1-trimethylolethane, or 1,1,1-trimethylolpropane; tetrols such as erythritol or pentaerythritol, pentols such as xylitol and ribitol, and higher polyols are useful in one or more reactions corresponding to those employed to make polyester II polyol adducts. In such embodiments, functionalization or polymerization of polyester II polyol adducts where more than two R⁸ moieties are hydrogen result in branched, hyperbranched, or dendritic structures.

In some embodiments, the polyesters II of the invention include compounds having the structure shown below:

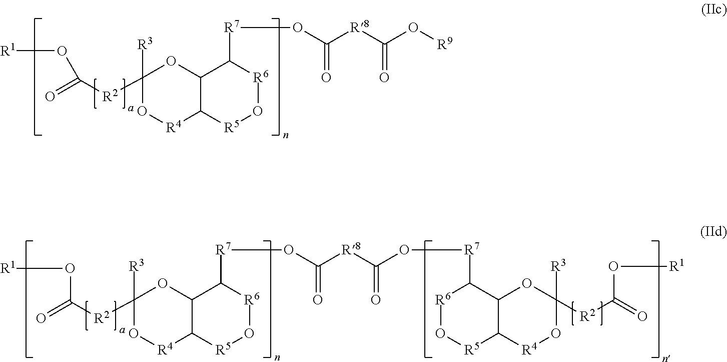

wherein R¹-R⁷ and a are as defined for compound I; n and n′ are between 1 and 10; and R′⁸ and R⁹ are linear, branched, or cyclic alkyl moieties or aromatic or alkyaromatic moieties having between 1 and 16 carbon atoms. These compounds are referred to herein as polyester II diester adducts. The polyester II diester adducts are formed by the reaction of compound I with the ester of a diacid such as any of those disclosed above, in conjunction with homopolymerization of compound I to give polyesters II. It will be appreciated by those of skill that adjustment of reaction conditions and stoichiometry are suitably varied to provide the desired amount of homopolymerization, that is, the desired values of n and n′. In other related embodiments, triacids such as trimellitic acid and cyclohexane tricarboxylic acid may be used in place of a diacids to form polyester II triester adducts analogous to the polyester II diesters adducts shown.

In embodiments, the polyester II diester adducts are plasticizers in a number of useful polymer compositions and, in some such embodiments, impart properties to the polymer that are similar to those imparted by the commercially available plasticizer dioctyl phthalate. Plasticizers are chemical compounds added to a base composition comprising one or more polymers with the purpose of lowering the glass transition temperature of the polymer composition, thereby making the composition more flexible and amenable to processing, e.g., by melt extrusion or molding. It will be understood that, depending on the polymer and the particular polyester II adduct used, other changes in physical and mechanical properties of the compounded polymer are conferred, as well as changes in barrier properties of the compounded polymer in respect to its permeability for various gases, water, water vapor, or organic compounds. It is also understood that, in various embodiments, one or more different polyester II diester adducts are employed as one part of a blend with additional plasticizers or other compounds for the preparation of an extrudable or moldable polymer compositions. Additional plasticizers include, for example, any of those commercially available compounds sold for plasticizing poly(vinyl chloride) or another polymer. Additional compounds include, in embodiments, various inorganic and organic filler compounds, wood dust, reinforcing fibers, crosslinkers, solvents, dyes, pigments, lubricants, anti-microbial or anti-fungal additives, thermal or UV stabilizers, and the like.

Polymers that are, in embodiments, plasticized by one or more polyester II diester adducts include, for example, poly(vinyl chloride), homopolymers and copolymers of polystyrene, poly(3-hydroxyalkanoates), poly(lactic acid), and various polysaccharide polymers, as well as polyesters II. Plasticizers are typically used at various effective concentrations that depend on the desired properties of the compounded polymer formulation. Polyester II diester adducts are incorporated, in some embodiments, at levels of between about 1% by weight and 80% by weight into a polymer.

In some embodiments, polyester II diester adducts are incorporated into a polymer by melt mixing, employing a temperature or range of temperatures that are above the melting point of the polymer. In some embodiments, polyester II diester adducts are introduced with a help of a solvent or as a component in a polymer plastisol, where it behaves both as a coalescing solvent and as a plasticizer. Many techniques for introducing plasticizer compounds to polymer compositions are known in the art and are suitably employed to incorporate polyester II diester adducts into one or more polymer compositions.

In embodiments, polyester II diester adducts are useful as monomers in one or more polymerization reactions. In some embodiments, polyester II diester adducts are diesters or diacids, and therefore are encompassed in one or more reactions to form polymeric compounds in any of the known reactions where diesters or diacids are employed. For example, linear polyesters or polyamides, or copolymers thereof, are formed by the reaction of polyester II diester adducts with diols, diamines, or aminoalcohols. Useful diols, diamines, or aminoalcohols include any of the diols, diamines, or aminoalcohols listed above and include, in some embodiments, compound I diols, compound I bis-diols, compound I aminoalcohols, and polyester II diol adducts described above. Branched and crosslinked compositions in conjunction with previously described polyfunctional compounds of the invention are also formed as described above. In some embodiments, the polyester II diester adducts are employed in a manner analogous to diester compounds described in Patent Application No. WO 2009/049041, the contents of which are incorporated herein by reference in their entirety.

In some embodiments, one or more polyester II diester adducts are reacted with one or more diamines to result in polyamide polymers. Such compounds are referred to herein as polyester II polyamides. The polyester II polyamide structures are formed using any of the diamine compounds listed above in conjunction with the polyester II diester adducts of the invention. In various embodiments, the polyester II polyamides are analogous to the polyamides formed from diesters and diamines as described in Canadian Patent Publication No. 2,676,898, the contents of which are incorporated herein by reference. As such, any of the methods employed to make polyamides disclosed in the reference are usefully employed to make polyester II polyamides of the current invention. In embodiments, one useful method for making the polyester II polyamides of the invention is to form a “nylon salt” of a diamine and the free acid of a polyester II diester adduct, in other words polyester II diester adducts wherein R¹ is hydrogen, followed by heating to form the corresponding polyester II polyamide. A stoichiometric balance of a free acid and diamine is achieved by forming the corresponding 1:1 ammonium salt in aqueous solution of about 10 wt % to 80 wt %, or about 50 wt %, of the combined free acid and diamine in water. Stoichiometry is achieved by controlling the pH of the solution by addition of the free acid or the diamine. Subsequent concentration of the salt to a slurry of about 60% by weight or greater is then achieved by removing some of the water at a temperature of about 100° C. or greater. Concentration is followed by polymerization by, in some embodiments, heating the concentrated slurry to about 200° C. or greater, or between about 200° C. and 250° C., or to about 210° C. During the polymerization, the temperature is, in some embodiments, raised to about 260° C. to 300° C., or to about 275° C. In some embodiments, a pressure of about 1.7 MPa or greater is employed during part of all of the polymerization reaction by allowing escape of water. In embodiments, no catalyst is required using this method.

In other embodiments, the reaction is carried out using a catalyst. In some such embodiments, 1,5,7-triazabicyclo[4.4.0]dec-5-ene or titanium tetraalkoxide are suitably employed as a catalysts that provides for the amidation to take place using mild conditions and resulting in high conversions of ester to amide moieties. In other embodiments, esters are reacted with amides without additional catalyst. In some embodiments, esters are reacted with amides at temperatures of about 150° to 200° C.

Copolyesters II and polyester II polyamides are, in various embodiments, random or segmented copolymers. For example, in some embodiments, copolyesters II and polyester II polyamides are prepared by copolymerization of pre-mixed monomers of compound I with one or or more other additional monomers, thereby resulting in random copolymers. In other embodiments, one or more homopolyesters II, copolyesters II, polyester II polyamides, or a combination thereof derived from compound I are prepared as described above. Subsequent to polymer preparation, additional monomers optionally including additional compounds I are added, and polymerization is continued. In such embodiments, segmented copolyesters II or segmented polyester II polyamides are formed. In such embodiments, in the a first polymerization, homopolyesters II, copolyesters II, or polyester II polyamides are prepared (“prepolymers II”), for example, to include one or more chains terminated with one or more hydroxyl groups, wherein the prepolymers II are linear or branched. The residual hydroxyl groups of prepolymers II serve as initiators for the second polymerization, which is carried out in the presence of one or more additional monomers. The second polymerization is optionally performed by polycondensation or by ring-opening polymerization processes such as those described elsewhere herein.

Ring opening polymerization of a hydroxyl-terminated prepolymer II is carried out, in embodiments, employing suitable amounts of a lactone, for example, lactide, thereby resulting in a segmented copolyester II comprising at least one segment containing polylactone and one segment containing prepolymer II. Such segmented copolyesters II are useful for making compatible blends including segmented polyesters II and one or more of any of the various polylactone homopolymers (e.g polylactic acid), thereby improving mechanical and heat-deflection properties of the polymer into which the segmented copolyester II or segmented polyester II polyamide is incorporated.

In a variation of such embodiments, the second polymerization is performed where two or more prepolymers are formed separately, then reacted together, wherein one or more prepolymers contain one or more residues of compound I. In some such embodiments, one or more of the prepolymers is an isocyanate-terminated prepolymer, such that the isocyanate-terminated prepolymer acts as a chain extender for a hydroxyl or an amino-terminated prepolymer. In such variations, the resulting segmented copolyester II or segmented polyester II polyamide chains (linear, or branched or crosslinked), can comprise multiple segments of each of the two or more various prepolymer segment types.

The various polyesters II and polyester II polyamides are, in embodiments, used in blends, optionally obtained by reactive extrusion. Blends include blends of various species of the polyesters II and polyester II polyamides, and other polymers incorporating compound I diols, compound I bis-diols, compound I aminoalcohols, polyester II diester adducts, and polyester II diol adducts of the invention as well as blends with such polymers as aliphatic/aromatic copolyesters, as for example polybutylene terephthalate adipate (PBTA), polybutylene terephthalate succinate (PBTS), and polybutylene terephthalate glutarate (PBTG); biodegradable polyesters such as polylactic acid, poly-ε-caprolactone, polyhydroxybutyrates such as poly-3-hydroxybutyrates, poly-4-hydroxybutyrates and polyhydroxybutyrate-valerate, poly-3-hydroxybutyrate-co-4-hydroxybutyrates, polyhydroxybutyrate-propanoate, polyhydroxybutyrate-hexanoate, polyhydroxybutyrate-decanoate, polyhydroxybutyrate-dodecanoate, polyhydroxy-butyrate-hexadecanoate, polyhydroxybutyrate-octadecanoate, and polyalkylene succinates and their copolymers with adipic acid, lactic acid or lactide and caprolactone and their combinations, and the like; polystyrene and copolymers thereof; polyurethanes; polycarbonates; polyamides such as Nylon 6 and Nylon 6,6; polyolefins such as polyethylene, polypropylene, and copolymers thereof; or any other industrially useful polymeric compounds. Blends also include, in some embodiments, composites with gelatinized, destructed and/or complexed starch, natural starch, flours, and other materials of natural, vegetable or inorganic origin. One or more polyesters II and polyester II polyamides of the invention are, in some embodiments, blended with polymers of natural origin, such as starch, cellulose, chitosan, alginates, natural rubbers or natural fibers (such as for example jute, kenaf, hemp). The starches and celluloses can be modified, such as starch or cellulose esters with a degree of substitution of between 0.2 and 2.5, hydroxypropylated starches, or modified starches with fatty chains, among others.

In embodiments, blends of polymers of the invention include blends of one or more polyesters II and polyester II polyamides with one or more impact modifiers. Impact modifiers are additives, often of a composite nature, that are added to a polymeric material to impart improved impact resistance. For example, core-shell acrylic impact modifiers such as those disclosed in U.S. Pat. Nos. 7,173,082 and 7,314,893 as well as any of the other known impact modifiers employed in the industry are useful in conjunction with the polyesters of the invention to impart improved impact resistance.

In embodiments, any of the compounds of the invention are tackifiers in one or more adhesive formulations. Tackifiers impart the sticky “feel” property familiar to users of pressure sensitive adhesive tapes, and are also incorporated into some hot melt adhesives. The polymers of the invention are tackifiers where, for example, a homopolyester II has a value of n of between 2 and 10. In embodiments where the compounds of the invention are employed as tackifiers, they impart aggressive adhesion to many different types of materials, including paper, metals, natural rubber, and synthetic polymers such as ethylene-vinyl acetate, styrene-butadiene block copolymers, styrene-isoprene block copolymers, and various acrylic polymers such as copolymers of iso-octyl acrylate, 2-ethyl hexyl acrylate, and acrylic acid.

The various compounds according to the invention, and blends of thereof, possess properties that render them suitable for use for numerous applications, by appropriately choosing chemical structures including stereoisomeric ratios, molecular weight, crosslink density, formulation components, and the like. Such applications include use as, or as a component of, one or more products such as films, fibers, injection-molded articles, extrusion coated articles, solution coated articles, foamed articles, thermoformed articles, extruded profiles and sheets, extrusion blow molded articles, injection blow molded articles, rotomolded articles, stretch blow molded articles, skived articles, milled articles, and the like. In the case of films, production technologies like film blowing, casting, and coextrusion can be used. Moreover such films can be subject to monoaxial or biaxial orientation in line or after film production. It is also possible that the stretching is obtained in presence of an highly filled material with inorganic fillers. In such a case, the stretching can generate micropores and the so obtained film can be suitable for hygiene applications.

The various polyesters II and polyester II polyamides according to the invention described above are suitable for the production of films. A “film” is defined, for the purposes of various embodiments of the invention, as a sheet type material that is flexible to e.g. bending and is between about 1 μm to 5 mm thick. Films may be made using one or more polyesters II and polyester II polyamides of the invention; or they can be made using another polymer blended with any of the compounds of the invention. Films employing various compounds and polymers of the invention are, in embodiments, one-directional or two-directional, single layer or multilayer, and employ one or more polyesters II or polyester II polyamides of the invention as a single component or in a blend with other materials, as described above. The films are useful for various applications including agricultural mulching films; printable films for graphics or text; cling films (extensible films) for foodstuffs, films for bales in the agricultural sector and for wrapping of refuse; shrink films such as for example for pallets, mineral water, six pack rings, and so on; bags and liners such as for collection of refuse, holding foodstuffs, gathering mowed grass and yard waste, and the like; thermoformed single-layer and multilayer packaging for foodstuffs, such as for example containers for milk, yogurt, meat, beverages, etc.; and in multilayer laminates with layers of paper, plastic materials, aluminum, and metalized films for a wide variety of applications.

The compounds of the invention as described above are also useful for coatings that form a layer on top of a film, an article, and the like. A coating may be up to several millimeters thick, or it may be a single molecular layer. Coatings of the invention are applied, in embodiments, by extrusion coating, die coating, slot coating, brush coating, spray coating, or any other generally known technique employed in the coating industry. Coatings employing various compounds and polymers made therefrom of the invention are useful as protective coatings, paint components, adhesives or glues, barrier layers, and the like. One or more coatings of the invention are applied, in embodiments, with or without additional solvent(s), such as coalescing solvents, and with our without additives such as UV blocking agents, antibacterial agents, colorants, fillers, and the like. One or more coatings of the invention are, in some embodiments, crosslinked after application.

The compounds of the invention, as included in one or more formulations, are also useful in forming articles. In some embodiments employing various polymers of the invention, articles are formed from the polymer alone without any additional components. An “article”, as defined for the purposes of the invention, includes objects that are be rigid or flexible; that exist as standalone objects or as part of an assembly or laminate; and that include one or more compounds and polymers made therefrom of the invention or a blend thereof, optionally with one or more additional materials. Some examples of useful articles that include various compounds and polymers made therefrom of the invention are punnets for foodstuffs, I-beams for construction, casings for e.g. pens, computer screens, and the like; parts for automobile construction, table tops, and the like; decorative items such as lamp parts, jewelry, vases, architectural features, and the like; children's toys; drink bottles; and many other articles. The invention is not particularly limited in terms of what articles may be formed employing the various compounds and polymers made therefrom of the invention. The articles comprising various polyesters II and polyester II amides can be filled with various organic and inorganic fillers. In such embodiments, non-limiting examples of inorganic fillers include fiberglass, silica, diatomaceous earth, gypsum, alkali-earth metal carbonates, alumosilicates, metal oxides such as oxides of aluminum, iron, titanium, clays, carbon black and the like. Examples of organic fillers include various plant fibers, ground corn cobs and corn fibers, wood dust, wood chips, straw, tree bark, oat hulls, and the like.

In embodiments employing the various polymers of the invention, articles are suitably formed wherein optical transparency coupled with shatter resistance and high strength are requirements in the application. For example, windows, such as projectile-deflecting optically transparent windows are suitably formed using various polymers of the invention. In such embodiments, the various polymers of the invention suitably replace commercially available polycarbonates containing Bisphenol A, wherein the bioactivity of Bisphenol A leacheates from polycarbonate has come under scrutiny. Further, the polyesters II of the invention are, in some embodiments, formed from 100% renewable sources rather than petroleum based compounds such as Bisphenol A. Due to their high strength and, in some embodiments, glass transition temperatures of over 100° C., the various polymers of the invention are usefully employed in applications involving elevated temperature. Thus, food containers that are microwavable or dishwasher safe are articles suitably formed from various polymers of the invention. Other suitable formulations and applications for various polymers of the invention include flexible cables; insulating film for magnet wire; medical tubing or other medical articles requiring autoclaving or subjection to sterilization temperatures or radiation; photoresist components; structural adhesive components; bushings, bearings, sockets or constructive parts in demanding applications such as where high temperatures or high stress is encountered; as a component of hot gas filters, e.g. in a nonwoven web; cases and housing for electronics such as computers and MP3 players; lenses for lighting or other optical apparatuses, such as streetlight lamp covers, eyeglass or sunglass lenses, or automotive headlamps; molded automotive parts such as steering wheels, instrument panel components or covers, interior molding, or exterior molded parts such as bumpers; riot gear such as shields, visors, and the like; children's toys such as spinning tops, RC cars, and the like; protective covers for posters and billboards, books or notebooks, and the like.

Articles that can be formed using formulations including one or more compounds of the invention include foamed articles. Art surrounding the foaming of polyurethanes is generally known in the industry are used, in embodiments, to form foamed articles from the various compounds and polymers made therefrom of the invention. Foamed articles include both rigid and flexible foams. Some examples of useful foamed materials include cushions for automobile seats, interior or exterior furniture, and the like; foamed or foamable beads for the production of pieces formed by sintering; foamed blocks made up of pre-foamed particles; foamed sheets, thermoformed foamed sheets, and containers obtained therefrom for the packaging of foodstuffs.

Articles also include fibrous articles. Examples of fibrous articles include standard scale fibers, microfibers, nanofibers, and composite fibers. Composite fibers have, in some embodiments, a core constituted by a rigid polymer such as PLA, PET, PTT, etc. and an external shell made with one or more polyesters II or polyester II polyamides of the invention; other composite fibers have various section configurations (from round to multilobed). Fibers also include flaked fibers, woven and non-woven fabrics or spun-bonded or thermobonded fabrics for the sanitary sector, the hygiene sector, the agricultural sector, georemediation, landscaping and the clothing sector.

Various embodiments are described in detail below. Reference to various embodiments does not limit the scope of the claims attached hereto. Additionally, any examples set forth in this specification are not intended to be limiting and merely set forth some of the many possible embodiments for the appended claims.

“About” modifying, for example, concentration, volume, process temperature, process time, yield, flow rate, pressure, the quantity of a compound or ingredient in a formulation or in an article, number of repeating organic units in a polymer, and like values, and ranges thereof, employed in describing the embodiments of the disclosure, refers to variation in the numerical quantity that can occur, for example, through typical measuring and handling procedures used for making compounds, compositions, concentrates, use formulations, or articles; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a reaction or a formulation with a particular initial concentration or mixture. Where modified by the term “about”, the claims appended hereto include equivalents to these quantities.

“Optional” or “optionally” means that the subsequently described event or circumstance may but need not occur, and that the description includes instances where the event or circumstance occurs and instances in which it does not. For example, “A optionally B” means that B may but need not be present, and the description includes situations where A includes B and situations where A does not include B.

“Includes” or “including” or like terms means “includes but not limited to.”

As used herein, the recitation in a claim of a claim element in the singular number is to be construed as not to exclude the presence of one or more of the same element.

As used herein, the term “ketal” means a cyclic, 5- or 6-membered acetal or ketal moiety or an acyclic acetal or ketal moiety, as indicated by one or more chemical structures described or shown. The term “ketalization” refers to a chemical reaction to form a cyclic, 5- or 6-membered acetal or ketal moiety or an acyclic acetal or ketal moiety.

As used herein, the term “carboxylate” means a carboxylic acid, a carboxylate salt, a carboxylate ester, or a carboxamide moiety, unless a specific compound having a carboxylic acid, a carboxylate salt, a carboxylate ester, or a carboxamide moiety as indicated by one or more chemical structures is described or shown.

As used herein the term “polymer” or “polymeric” encompasses any reaction product wherein condensation or addition reaction results in the formation of more than one repeating organic unit. Thus, “polymer” or “polymeric” encompass dimers, trimers, tetramers, and higher numbers of repeating units, oligomers, and the like up to and including compounds having hundreds or thousands of repeating organic units. The repeating organic units may be the same or different as indicated by one or more chemical structures is described or shown.

The compounds of the invention have, in embodiments, one or more isomers. Where an isomer can exist but is not specified, it should be understood that the invention embodies all isomers thereof, including stereoisomers, conformational isomers, and cis, trans isomers; isolated isomers thereof and mixtures thereof.

The present invention may suitably comprise, consist of, or consist essentially of, any of the disclosed or recited elements. Thus, the invention illustratively disclosed herein can be suitably practiced in the absence of any element which is not specifically disclosed herein.

### EXPERIMENTAL SECTION

The following Examples further elucidate and describe the compounds of the invention and applications thereof without limiting the scope thereof. The graphical representations of the reactions carried out in the Examples are meant to be illustrative of the chemical reactions conducted and are not meant to limit the scope of possible products formed thereby.

### General Information

Unless specified otherwise below, all chemicals, reagents and solvents used in the foregoing examples were purchased from the Sigma Aldrich Company of St. Louis, Mo., USA and were at least of 99% purity.

Ethyl levulinate (99%+ purity) and butyl levulinate (99%+ purity) were purchased from Langfang Triple Well Chemicals Company, Ltd. of Langfang City, HeBei, China.

Xylitol of 99%+ purity (FCC grade) was purchased from Epic Industries, Inc, of Provo, Utah, USA.

### Example 1

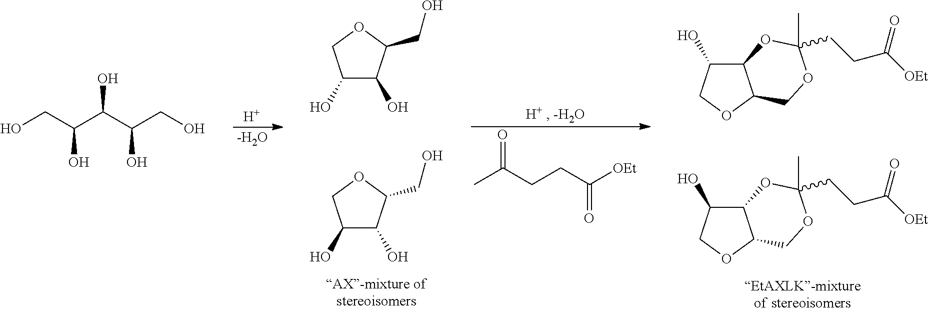

A 500 mL round-bottomed flask was charged with 50.42 g (0.331 mol) xylitol and 6.8 μL (0.128 mmol) sulfuric acid. The flask was heated on a Kugelrohr apparatus at 160° C. (air oven temperature). Water formed during the reaction was removed under vacuum (5-10 Torr) and collected in a dry ice-isopropanol cold trap. After 6 hours, the reaction flask was taken off of the Kugelrohr apparatus and allowed to cool to ambient temperature. Then the reaction flask was charged with 190.47 g (1.321 mol) ethyl levulinate, and equipped with a Dean-Stark trap, condenser, and magnetic stir bar. The reaction flask was heated in a 110° C. oil bath for 2 hours under vacuum (40-50 Torr). The reaction flask was then taken out of oil bath and allowed to cool to ambient temperature.

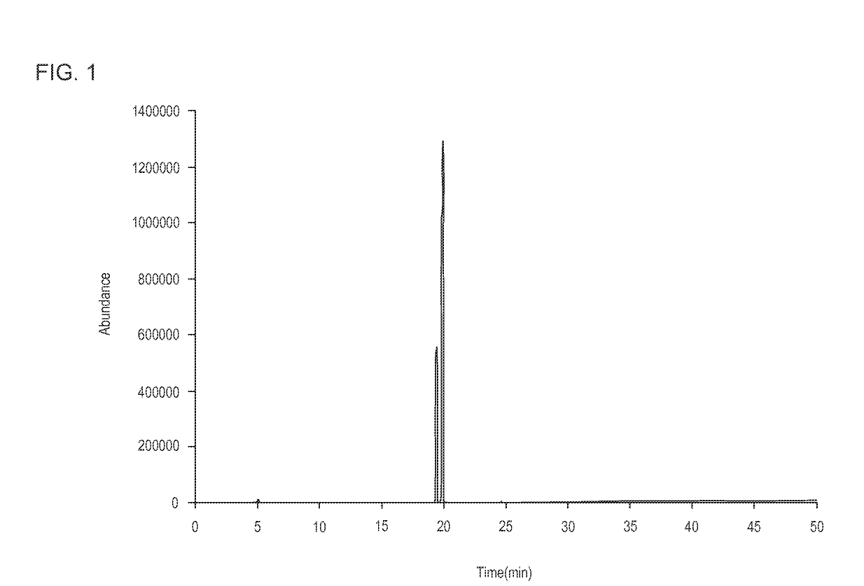

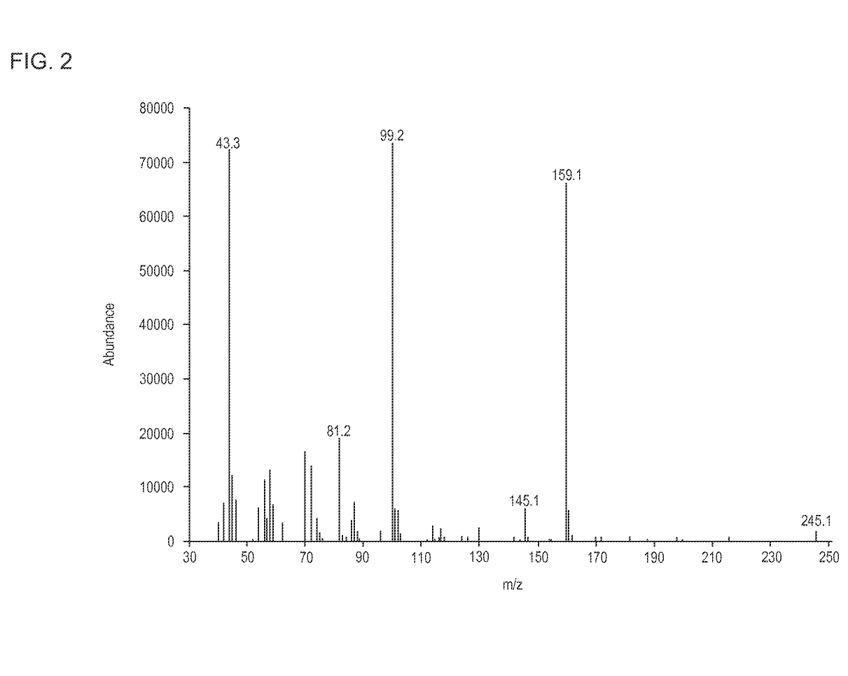

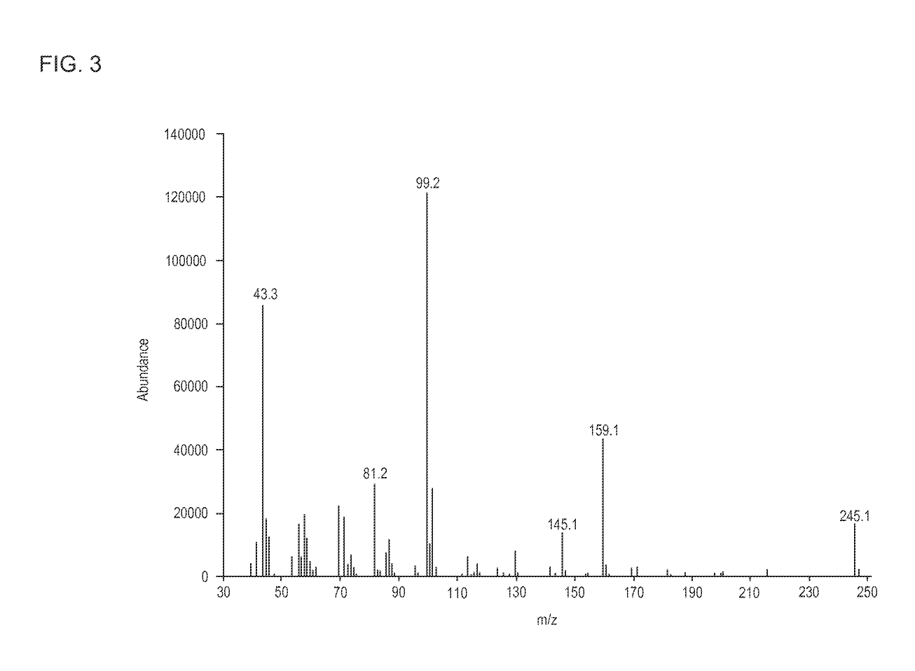

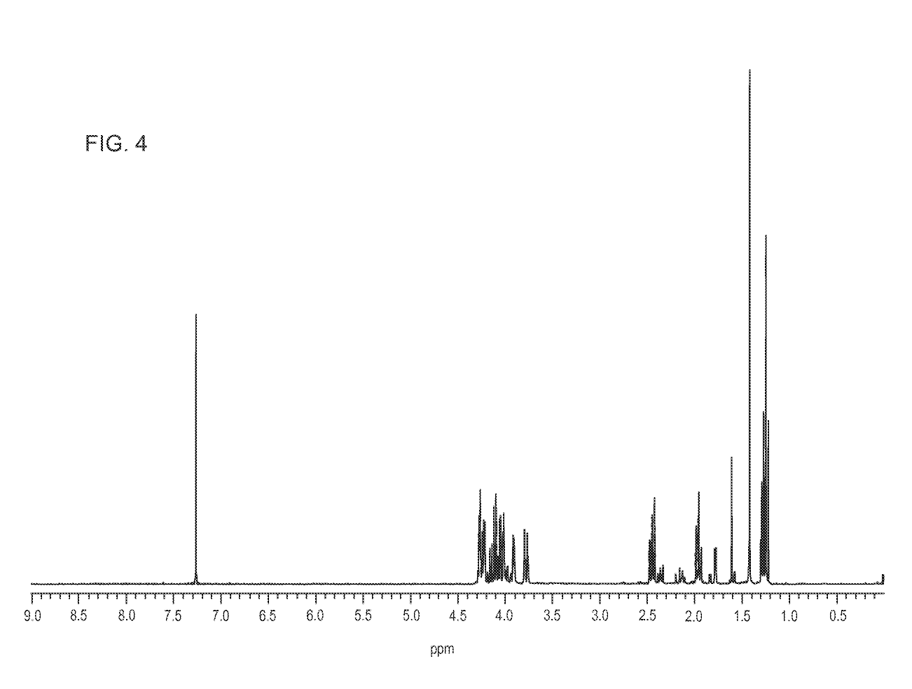

The reaction mixture was diluted with 500 mL ethyl acetate, washed with saturated sodium bicarbonate three times (300+200+200 mL) and saturated sodium chloride solution one time (300 mL). The organic layer was dried over anhydrous sodium sulfate. After filtering off the solids, the filtrate was concentrated on a rotary evaporator to remove ethyl acetate. The residue was further distilled on Kugelrohr apparatus, first to remove unreacted ethyl levulinate, then to distill the desired reaction product as a very pale green viscous liquid at 190° C. under 200 mTorr vacuum. The viscous liquid solidified very quickly to form a creamy white, crystalline appearing solid during the distillation. The solid was analyzed by GC-MS and ¹H NMR to confirm the structure as that of the 1,4-anhydroxylitol levulinate ketal, ethyl ester (EtAXLK). Total yield of EtAXLK crystal obtained was 38.58 g (44.7 mol % yield from xylitol). GC-MS Total Ion Chromatogram of EtAXLK, shown in FIG. 1, showed purity was >99.5%. The mass spectra of the two isomers are shown in FIG. 2 and FIG. 3, respectively. The NMR spectrum is shown in FIG. 4.

### Example 2

The synthesis of EtAXLK was carried out employing a different technique than that used in Example 1. A 1 L 3-neck flask equipped with a mechanical stirrer and a thermocouple was charged with 152.35 g (1.00 mol) xylitol (obtained from the Sigma-Aldrich Company of St. Louis, Mo.) and 20 μL (0.376 mmol) concentrated sulfuric acid The flask was heated with heating mantle until the temperature of the material in the flask reached 160° C. Water was removed from the reaction flask under vacuum (about 10 Torr) and collected in a dry ice-isopropanol cold trap. After 6 hours, the reaction flask was backfilled with nitrogen and then charged with 576.10 g (4.00 mol) ethyl levulinate The reaction mixture was heated to 110° C. and maintained at that temperature for 2 hours under vacuum (about 40 Torr). The heating mantle was then removed and the reaction flask was allowed to cool to ambient temperature.

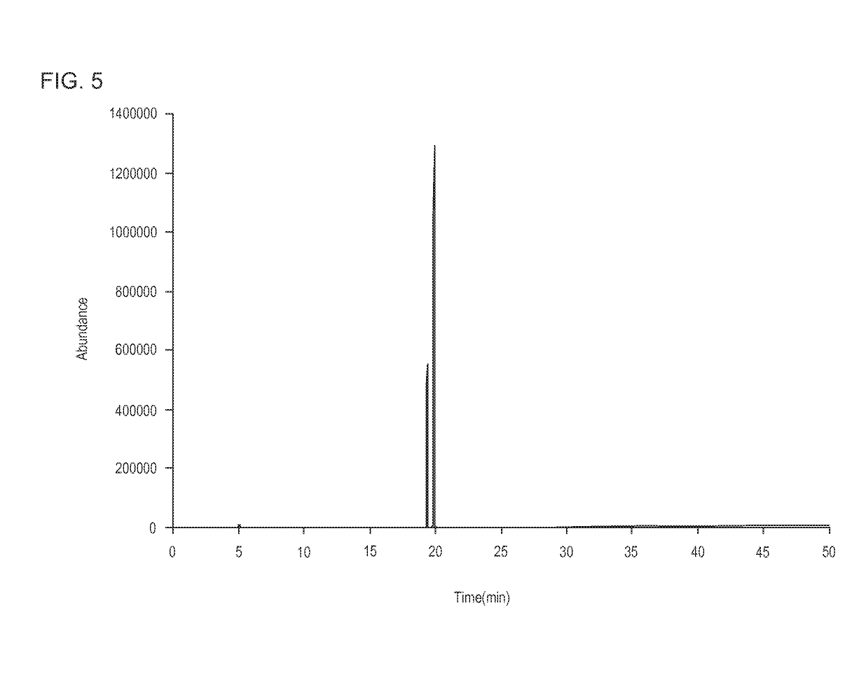

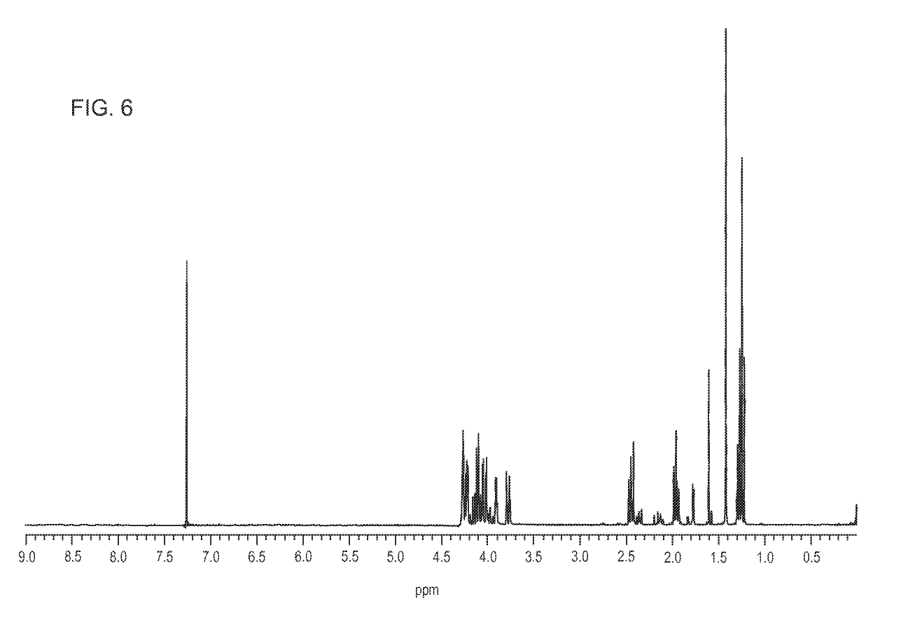

The reaction mixture was diluted with 1 L ethyl acetate, washed four times with 300 mL of a saturated sodium bicarbonate solution and once with 300 mL of saturated sodium chloride solution. The organic layer was collected and dried over anhydrous sodium sulfate. After filtering off the solid, the filtrate was concentrated on a rotary evaporator to remove ethyl acetate and some unreacted ethyl levulinate. The residue was further distilled on a Kugelrohr apparatus, to remove ethyl levulinate, then to distill off a material that immediately formed creamy white crystals at 185-190° C. under 200 mTorr vacuum. The crystals were analyzed by GC-MS and ¹H NMR to confirm the structure as EtAXLK. Total amount of EtAXLK crystal obtained was 105.35 g (40.4 mol % yield from xylitol). The GC-MS Total Ion Chromatogram, shown in FIG. 5, showed purity >99.5%. The NMR spectrum is shown in FIG. 6.

### Example 3

### A. General Procedure for the Preparation of Crude 1,4-anhydroxylitol (Xylitan)

A 2-liter round bottom flask is charged with 1 kg of solid xylitol and pre-measured catalytic amounts of concentrated sulfuric acid pre-dissolved in 0.5 or 1 mL of deionized water. The total amount of sulfuric acid in the water is adjusted to be in the range of about 50 ppm to 200 ppm based on the total weight of reaction mixture. The flask is then attached to a rotary evaporator equipped with an oil bath pre-heated to a temperature of 170° C., and a vacuum of about 20 Torr is applied to the flask. The evacuated flask is rotated in the oil bath. After about 30 minutes, the contents of the flask melt and distillation of water commences. The water is collected to a graduated cylinder. Collection is continued until about 120 mL of water is collected, at which point the reaction time is recorded and the flask is removed from the oil bath and allowed to cool to room temperature.

The resulting crude reaction product is a yellow to brown transparent liquid, wherein lesser amounts of sulfuric acid result in lighter colored crude reaction product. Where 50 ppm of sulfuric acid is used, the crude reaction product is nearly colorless. The contents of the flask are weighed to determine crude reaction product weight. The crude reaction product is derivatized by acetylation with acetic anhydride/pyridine using known techniques, and the derivatized crude reaction product is analyzed by GC-MS.

Some actual reaction conditions, times, and crude product weight employing the general procedure are shown in Table 1. The crude reaction product obtained using this general reaction procedure typically comprises about 90% of 1,4-anhydroxylitol (xylitan, racemic mixture), about 3% of other anhydroxylitol isomers, including 1,5-anhydroxylitol, about 0.7-1.5% of unreacted xylitol, and about 5-6% of oligomeric products.

TABLE 1
Reaction conditions of some reactions
run according to procedure 3A.

| | | | | Crude |
|---|---|---|---|---|
| | H₂SO₄ | Temperature | Reaction | product wgt % |
| Reaction | conc (ppm) | (° C.) | time (min) | (based on xylitol) |
| 3A-1 | 92 | 160 | 300 | 87.1 |
| 3A-2 | 92 | 170 | 135 | 86.3 |
| 3A-3 | 92 | 180 | 105 | 85.3 |
| 3A-4 | 184 | 160 | 180 | 87.1 |
| 3A-5 | 184 | 170 | 114 | 86.5 |
| 3A-6 | 50 | 170 | 200 | 87.7 |

### B. General Procedure for Purification of 1,4-anhydroxylitol by Distillation

A 1 L round bottom flask is charged with 300-500 g of the crude reaction product prepared according to general procedure 3A. Then about 300-500 mg of sodium carbonate pre-dissolved in 1-2 ml of deionized water is added to the flask. The flask is attached to a rotary evaporator equipped with an insulated covered oil bath set at 110° C., and the flask is rotated while a 20 Torr vacuum applied to the flask for about 1 hour. Then pressure is reduced further in the flask. After pressure is stabilized at about 1-2 Torr, the temperature in the oil bath is gradually increased to 180° C. over about 1 hour. Distillation of a colorless or slightly yellow liquid is observed to start at about 0.5-1 Torr, and oil bath temperature of about 180° C. The liquid is collected into a 500 ml flask equipped with splash guard until distillation subsides. The distilled liquid accounts for about 77-85 wt % of crude reaction product. The liquid is used in the subsequent examples without any additional purification.

### Example 4

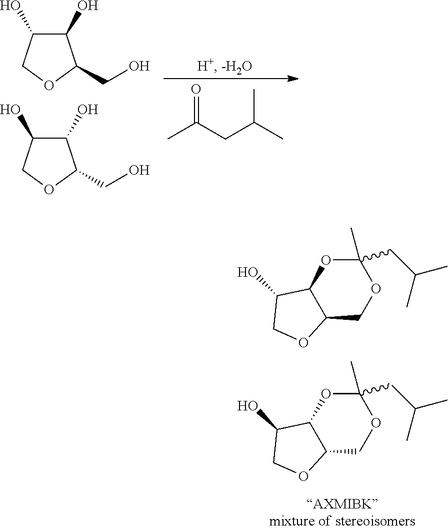

A 2 L round-bottomed 4-neck flask was charged with 200.6 g of crude anhydroxylitol obtained according to Example 3A and containing estimated 55 mg sulfuric acid (based on the amount used in the preparatino of anhydroxylitol) and 601.0 g of methyl isobutyl ketone, or MIBK. The flask was equipped with a mechanical stirrer, a thermocouple, a nitrogen outlet and a Dean-Stark trap and condenser. Using a heating mantle, the contents of the flask were heated to reflux temperature (about 116°-120° C.) under a nitrogen blanket. Upon reaching reflux in the flask, liquid was observed to collect in the Dean Stark trap. Reflux was continued until liquid stopped collecting in the trap, about 16 hours. The flask was then allowed to cool to room temperature.

Upon cooling, the reaction mixture formed two observable layers. The layers were separated, and the lower layer (32.9 g) was discarded. The upper layer (710.1 g) was analyzed by GC-MS and was found to contain the 1,4-anhydroxylitol methyl isobutyl ketal (AXMIBK) and MIBK. About 1 g of Na₂HPO₄ was added to the contents of the upper layer and this mixture was stirred at ambient temperature for about 80 minutes. Then the solids were filtered from the liquid and the solids were discarded. The filtered liquid was stripped of MIBK using a rotary evaporator under reduced pressure. The stripped liquid was distilled using a Kugelrohr apparatus at about 250-300 mTorr and about 160° C. to yield 191.95 g of pale yellow, viscous liquid. The distilled liquid was determined by GC-MS to be 98% AXMIBK as a 1:2 mixture of cis:trans isomers. The cis:trans ratio herein and elsewhere in the Examples was determined on the basis of integration of fully separated peaks in GC-TIC chromatogram. The principal impurity, about 1.8% by GC-MS, was determined to be unreacted anhydroxylitol isomers.

The distilled liquid was dissolved in about 600 mL of methyl t-butyl ether and washed twice with 30 mL of 10% aqueous solution of sodium carbonate. The organic layer was collected and dried over anhydrous sodium sulfate, filtered, and the solvent was removed under reduced pressure on a rotary evaporator to yield a final product. The final product (182.2 g) was >99.6% AXMIBK by GC-MS and contained no detectable amounts of unreacted anhydroxylitol isomers.

### Example 5

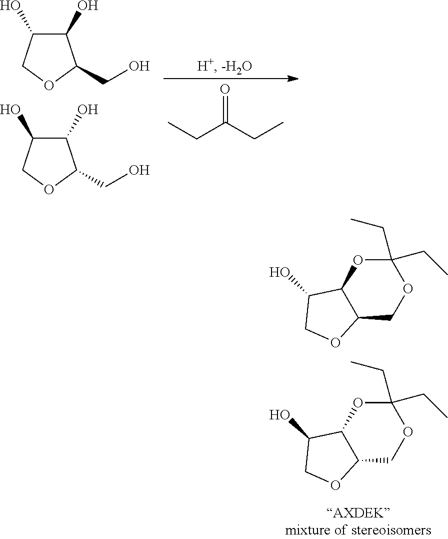

A 500 mL, 3-neck round bottom flask was charged with 150 g (1.74 mol) 3-pentanone (diethyl ketone, or DEK) and 48.8 g (0.36 mol) crude anhydroxylitol prepared according to the Example 3A. The contents of the flask formed two observable liquid layers. The flask was equipped with an overhead mechanical stirrer, nitrogen inlet, and Dean-Stark trap with an overhead condenser. The flask was and immersed in an oil bath set to a temperature of about 120° C. The contents of the flask were blanketed with a nitrogen stream and heated to about 120°-134° C. Upon reaching this temperature range, liquid was observed to collect in the Dean Stark trap. After about 9 hours, liquid collection in the trap subsided, and the contents of the flask were allowed to cool to room temperature. A sample of the cooled flask contents was removed for GC-MS analysis. The analysis showed that the flask contents contained approximately 90% 1,4-anhydroxylitol diethyl ketal (AXDEK).

About 1 g sodium bicarbonate was added to the flask, and the contents of the flask were stirred for about 60 minutes at room temperature. Then the contents of the flask were added to a 500 mL single neck flask equipped with a fractionation column, condenser, vacuum/nitrogen inlet, and an adapter with a set of receiving flasks. The flask was immersed in an oil bath set to a temperature of about 200° C. and the contents of the flask were vacuum distilled at about 2-3 Torr, where in the major fraction of distillate was observed to distil at a head temperature of about 158°-160° C. About 43.6 g of a nearly colorless, transparent liquid was collected that slowly crystallized on standing. The major fraction was confirmed by GC-MS to be 98% AXDEK.

### Example 6

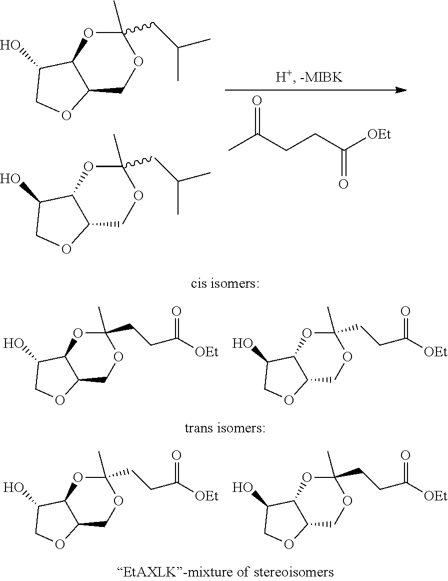

A 20 mL vial was equipped with a stir bar, and 5.0 g (0.03 mol) ethyl levulinate, 2.2 g (0.01 mol) AXMIBK prepared according to the procedure of Example 4, and 0.5 mg p-toluenesulfonic acid monohydrate were added to the vial. The contents of the vial were stirred at room temperature (about 21° C.) and aliquots were periodically removed for electron-ionization GC-MS analysis. Prior to mixing the reagents in the vial, ethyl levulinate and AXMIBK were analyzed by GC-MS to provide the t=0 starting point basis for monitoring the reaction to form EtAXLK. The residual AXMIBK stereoisomers were separately detected and analyzed to determine percent conversion on the basis of integration of fully separated isomer peaks.

As the reaction proceeded, the EtAXLK trans and cis stereoisomer pairs were detected as fully separated peaks on and identified on the basis of their mass spectra and retention times, and quantified by integrating GC-TIC chromatograms. After about 24 hours, stirring was stopped and a rapid solidification that appeared to be crystallization immediately occurred in the reaction mixture. The mother liquor (approximately 3.1 g) and wet product crystals (approximately 3.9 g) were separated by decanting and analyzed separately by GC-MS. The results are summarized in Table 2.

TABLE 2
Conversion, product stereoisomer ratio as measured by GC-MS.

| Time | % Conversion | EtAXLK, trans:cis |
|---|---|---|
| (hours) | (by TICof GC-MS) | (X trans:1 cis) |
| 0 | 0 | n/a |
| 0.5 | 16 | 6.96 |
| 1.7 | 50 | 5.73 |
| 3.0 | 65 | 5.17 |
| 7.0 | 94 | 4.35 |
| 24.0 | 99+ | 4.02 |
| | | (mother liquor) |
| | | 23 |
| | | (wet crystals) |

### Example 7

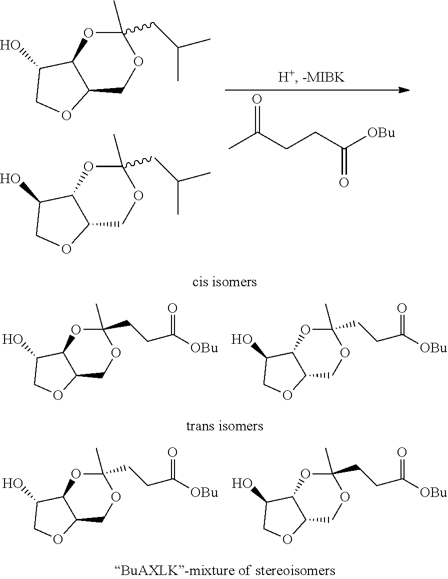

A reaction was carried out according to the procedure of Example 6, except that 5.05 g (0.03 mol) of butyl levulinate was used instead of ethyl levulinate.

After 24 hours, a 99+% conversion of AXMIBK to BuAXLK was measured using the GC-MS techniques described in Example 6. No crystallization was observed to occur in the reaction mixture. The final product had a 4.02:1 ratio of trans/cis isomers.

### Example 8

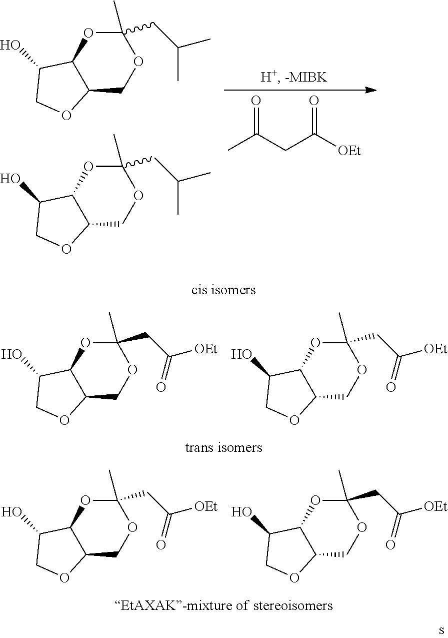

A reaction was carried out according to the procedure of Example 6, except 4.96 g (0.04 mol) of ethyl acetoacetate was used instead of ethyl levulinate. After 24 hours, a 97% conversion of AXMIBK to EtAXAK was measured using the GC-MS techniques described in Example 6. No crystallization was observed to occur in the reaction mixture. The final product had a 2.73:1 ratio of trans:cis isomers.

### Example 9

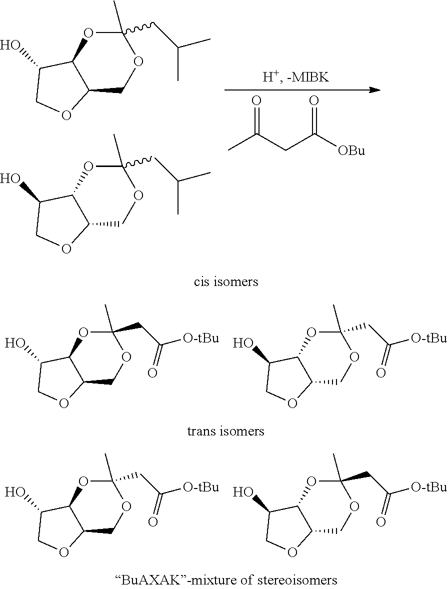

A reaction was carried out according to the procedure of Example 6, except 5.07 g (0.03 mol) of t-butyl acetoacetate was used instead of ethyl levulinate. After 24 hours, a 96% conversion of AXMIBK to BuAXAK was measured using the GC-MS techniques described in Example 6. No crystallization was observed to occur in the reaction mixture. The final product had a 2:1 ratio of trans:cis isomers.

### Example 10

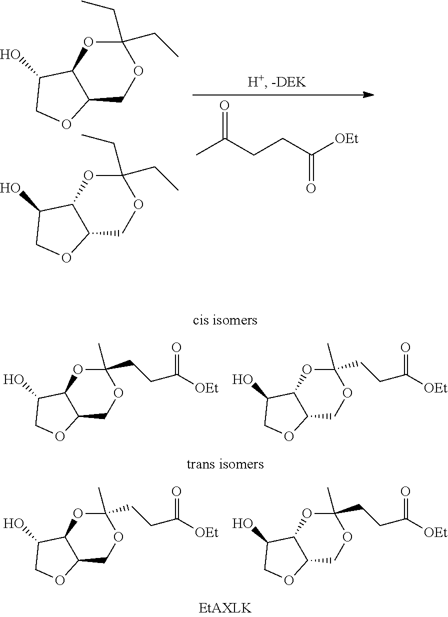

A 20 mL vial was equipped with a stir bar, and 6.0 g (0.04 mol) ethyl levulinate, 2.0 g (0.01 mol) of AXDEK prepared according to the Example 5, and 1.0 mg p-toluenesulfonic acid monohydrate were added to the vial. The contents of the vial were stirred at room temperature (about 21° C.) and aliquots were periodically removed for electron-ionization GC-MS analysis. Prior to mixing the reagents in the vial, ethyl levulinate and AXDEK were analyzed by GC-MS to provide the t=0 starting point basis for monitoring the reaction to form EtAXLK.

After 15 hours, 95% conversion of AXDEK to EtAXLK was measured using the GC-MS techniques described in Example 6. No crystallization was observed to occur in the reaction mixture. The final product had a 3.73:1 ratio of trans:cis isomers.

### Example 11

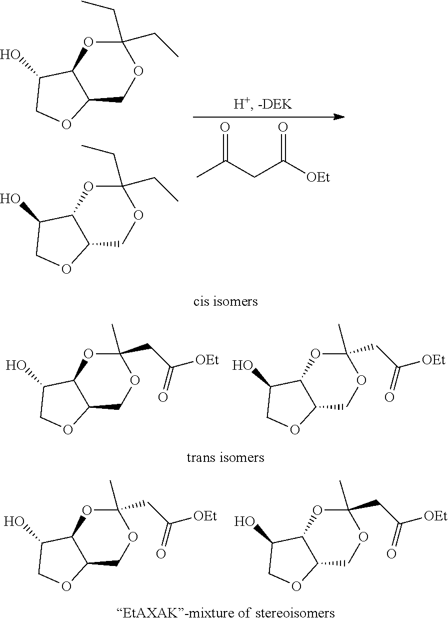

A reaction was carried out according to the procedure of Example 10, except 6.0 g (0.05 mol) of ethyl acetoacetate was used instead of ethyl levulinate. After 15 hours, a 37% conversion of AXDEK to EtAXAK was measured using the GC-MS techniques described in Example 6. No crystallization was observed to occur in the reaction mixture. The product had a 2.5:1 ratio of trans:cis isomers.

### Example 12

A reaction was carried out according to the procedure of the Example 6, except that 5.0 g (0.04 mol) ethyl pyruvate was used instead of ethyl levulinate. No reaction was observed after 24 and 48 hours of stirring at room temperature.

### Example 13

A reaction was carried out according to the procedure of the Example 6, except that 5.3 g (0.02 mol) butyl 2,2-dibutoxyacetate (glyoxylic acid butyl ester dibutyl acetal) was used instead of ethyl levulinate. No reaction was observed after 24 and 48 hours of stirring at room temperature.

### Example 14

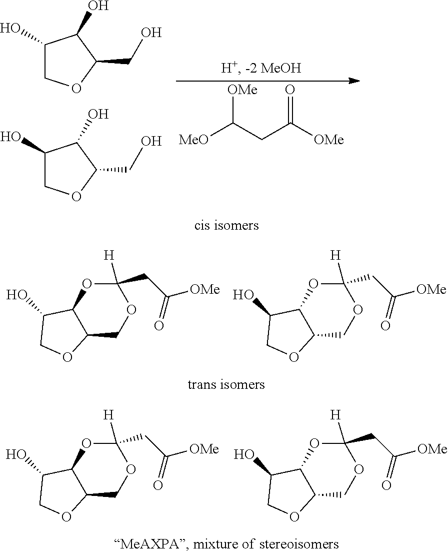

A 20 mL vial was equipped with a stir bar, and 5.06 g (0.03 mol) methyl 3,3-dimethoxypropionate (dimethyl ketal of methyl formylacetate or methyl 3-oxopropionate), 1.8 g (0.01 mol) anhydroxylitol prepared according to the procedure of Example 3B, 1.2 g (0.02 mol) of acetone), and 1.0 mg p-toluenesulfonic acid monohydrate were added to the vial. The contents of the vial were stirred at room temperature (about 21° C.) and aliquots were periodically removed for analysis using the GC-MS techniques described in Example 6.

After 24 hours, the contents of the vial were found to contain 26% of anhydroxylitol dimethyl ketal (reaction product of 1,4-anhydroxylitol and acetone), 19% 1,4-anhydroxylitol 3-oxopropropionate acetal (MeAXPA), present as a 1.1:1 trans:cis ratio, and approximately 40% of a complex mixture of acyclic acetal products resulting from partial acetal exchange between hydroxyl groups of 1,4-anhydroxylitol and the acetal group of methyl 3,3-dimethoxypropionate.

### Example 15

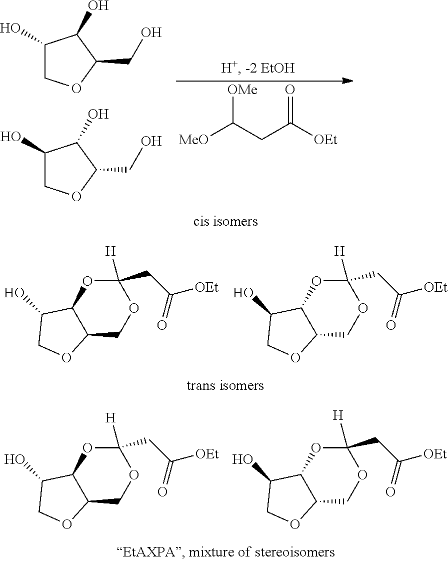

A reaction was carried out according to the procedure of Example 14, except that 5.0 g (0.03 mol) ethyl 3,3-dimethoxypropionate was used instead of methyl 3,3-dimethoxypropionate, and 1.9 g (0.01 mol) 1,4-anhydroxylitol was used in the reaction mixture.

After 24 hours, the contents of the vial were found to contain 23% of anhydroxylitol dimethyl ketal, 14% EtAXPA, present as a 1.3:1 trans:cis ratio, and approximately 52% of a complex mixture of acyclic acetal products resulting from partial acetal exchange between hydroxyl groups of 1,4-anhydroxylitol and the acetal group of ethyl 3,3-dimethoxypropionate.

### Example 16

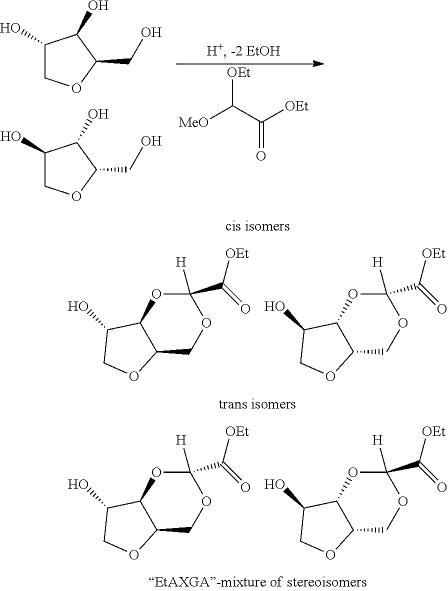

A reaction was carried out according to the procedure of Example 15, except that 5.1 g (0.03 mol) ethyl 2,2-diethoxyacetate was used instead of ethyl 3,3-dimethoxypropionate. After 24 hours, the contents of the vial were found to contain 27% of 1,4-anhydroxylitol dimethyl ketal, 17% 1,4-anhydroxylitol glyoxylate acetal, ethyl ester (EtAXGA), present as a 1:1 trans:cis ratio, and approximately 46% of a complex mixture of acyclic acetal products resulting from partial acetal exchange between hydroxyl groups of 1,4-anhydroxylitol and the acetal group of ethyl 2,2-diethoxyacetate.

### Example 17

A 1-liter 3-neck round bottom flask was charged with 386 g (2.24 mol) n-butyl levulinate, 150 g (1.12 mol) of 1,4-anhydroxylitol prepared according to the Example 3B, and 5.4 mg (0.055 mmol) of 98% sulfuric acid. The contents of the flask were observed to form two distinct liquid layers. The flask was equipped with an overhead mechanical stirrer, a Dean-Stark separator with an overhead condenser and vacuum/nitrogen inlet, and thermocouple. The contents of the flask were heated to 90° C. by means of an oil bath, under reduced pressure of about 9-12 Torr while stirring for approximately 40 minutes, and a liquid was collected in the Dean Stark trap. After collection of liquid subsided, the contents of the flask were allowed to cool to room temperature, and a sample of the crude product was removed for GC-MS analysis. The analysis showed that the crude product was about 42.1% n-butyl levulinate, about 1.6% 1,4-anhydroxylitol, and about 55.3% 1,4-anhydroxylitol levulinate ketal, butyl ester (BuAXLK).

The crude product was washed once with an equal volume of a 1 wt % aqueous solution of sodium carbonate and then twice with an equal volume of 0.2 wt % aqueous sodium bicarbonate solution in a separation funnel. A sample of the washed product was removed for GC-MS analysis. The GC trace showed that the unreacted 1,4-anhydroxylitol was completely removed. Residual butyl levulinate and any traces of water were distilled out under reduced pressure using a rotary evaporator (170° C., 20 Torr). The resulting liquid was distilled using a rotary evaporator using an oil bath set to 170°-175° C. and reduced pressure of 0.2-0.6 Torr. The resulting distilled colorless liquid product (155 g) was analyzed by GC-MS and was found to be over 99% 1,4-anhydroxylitol levulinate ketal, butyl ester (BuAXLK), present as a 3:1 mixture of trans:cis isomers.

### Example 18

A 1-liter 3-neck round bottom flask was charged with 461 g (3.55 mol) ethyl acetoacetate, 130 g (0.97 mol) 1,4-anhydroxylitol, prepared according to the procedure of Example 3B, and 8.7 mg (0.089 mmol) of 98% sulfuric acid. The contents of the flask were observed to form two distinct liquid layers. The flask was equipped with an overhead mechanical stirrer, a Dean-Stark separator with an overhead condenser and vacuum/nitrogen inlet, and thermocouple. The contents of the flask were heated in an oil bath to about 90° C. at reduced pressure of about 20-50 Torr while stirring for approximately 2 hours. During this time, a liquid was observed to collect in the Dean Stark trap. The distillate was observed to separate into two layers as it cooled. After 2 hours, the pressure in the flask was lowered to about 7-13 Torr in order to strip off remaining ethyl acetoacetate. A liquid was observed to distill from the flask. When liquid stopped collecting, the flask was cooled to room temperature.

A sample of the reaction mixture was removed for GC-MS analysis. The analysis showed that the crude product contained 95% 1,4-anhydroxylitol acetoacetate ketal, ethyl ester (EtAXAK). The crude product was further purified by subjecting to vacuum distillation on a rotary evaporator while immersed in an oil bath set to about 160° C. and at reduced pressure of about 0.4-0.6 Torr. About 173 g of distillate was collected. The distilled product was 96% EtAXAK, present as a 0.85:1 mixture of trans:cis isomers.

### Example 19

A 1 liter, 3-neck round bottom flask was charged with 243 g (2.09 mol) methyl acetoacetate, 80 g (0.60 mol) 1,4-anhydroxylitol prepared according to the procedure of Example 3B, and 3.2 mg (0.033 mmol) of 98% sulfuric acid. The contents of the flask were observed to form two distinct liquid layers. The flask was equipped with a mechanical stirrer, a Dean-Stark trap with an overhead condenser, and a thermocouple. The contents of the flask were heated to 100° C. using an oil bath under reduced pressure of 50-100 torr while stirring for approximately 1 hour. During this time, a distillate was collected in the Dean Stark trap. The distillate separated into two layers as it was cooled. After 1 hour, the pressure in the flask was lowered to about 5-25 Torr in order to strip off remaining methyl acetoacetate. A liquid was observed to distill from the flask. When liquid stopped collecting, the flask was cooled to room temperature.

A sample of the reaction mixture was removed for GC-MS analysis. The analysis showed that the reaction product contained about 90% anhydroxylitol acetoacetate ketal, methyl ester (MeAXAK). The reaction product was further purified by subjecting to vacuum distillation using a rotary evaporator with an oil bath set at 155° C., at reduced pressure of about 0.4-0.8 ton. About 112 g of distillate was collected. The distilled product was about 93% MeAXAK, present as a 0.92:1 mixture of trans:cis isomers.

### Example 20

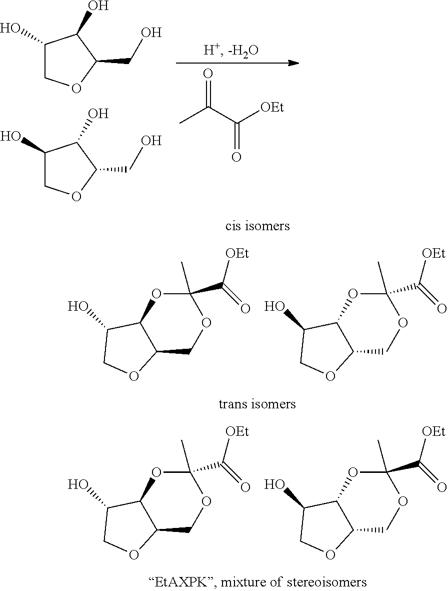

A 1-liter 3-neck round bottom flask was charged with 261 g (2.31 mol) ethyl pyruvate, 50 g (0.37 mol) 1,4-anhydroxylitol prepared according to the procedure of Example 3B, 122 g (1.45 mol) cyclohexane and 11.2 mg (0.11 mmol) of 98% sulfuric acid. The contents of the flask were observed to form two distinct liquid layers. The flask was equipped with an overhead mechanical stirrer, a Dean-Stark separator with an overhead condenser and vacuum/nitrogen inlet, and a thermocouple. The contents of the flask were heated to about 100°-110° C. using an oil bath while stirring for approximately 2 hours, and a distillate was collected in the separator. After 2 hours, the pressure in the flask was reduced to about 300-400 Torr for about 4 hours to strip the reaction mixture of cyclohexane and excess ethyl pyruvate. The flask was allowed to cool to room temperature when liquid stopped collecting in the trap.

A sample of the product mixture was removed for GC-MS analysis. The analysis showed that the product mixture contained about 80% 1,4-anhydroxylitol pyruvate ketal, ethyl ester (“EtAXPK”). The reaction product was subjected to vacuum distillation at 150° C. at about 0.6-0.7 Torr. About 27.9 g of distillate was collected. Analysis by GC-MS showed the purity of distilled EtAXPK was >96%.

### Example 21

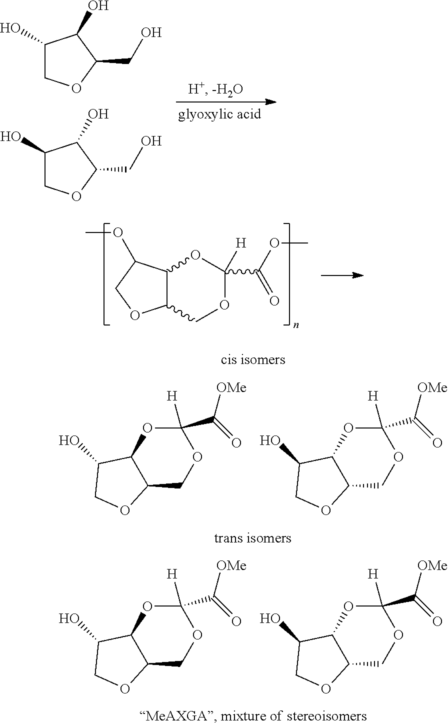

A 500 mL, single neck round bottom flask was charged with 31.3 g (0.23 mol) distilled 1,4-anhydroxylitol prepared according to the procedure of Example 3B, 35.4 g of 50 wt % solution of glyoxylic acid in water, and 0.025 mL of deionized water to form a homogeneous, colorless mixture. The flask was attached to a rotary evaporator and was rotated at about 50 rpm under reduced pressure of about 15 Torr while immersed in an oil bath set to 80° C. A liquid was observed to collect in the catch flask of the evaporator. The temperature of the oil bath was gradually increased to about 140° C. over period of about 4 hours. The contents of the flask solidified during this time and did not flow even when the temperature of oil bath was raised to 180° C. for 1 hour. The resulting product was 43.1 g of an amber transparent brittle solid that was soluble in water and insoluble in tetrahydrofuran or butanol. DSC was used to measure the glass transition temperature of the polymer which was 88° C.

A 1.2 g portion of the crude reaction product was mixed with 10 mL of methanol containing 50 mg of sodium methoxide, and the mixture was stirred by means of magnetic stirring for 12 hours at room temperature until completely dissolved. A 0.25 mL aliquot of the resulting solution was mixed with 1.5 mL of methyl t-butyl ether, and 0.05 mL of acetic acid was added; the insoluble matter was precipitated by centrifugation. The resulting clarified solution (about 1.7 ml) was collected and was found to contain approximately 5 mg of dissolved matter after evaporation of the solvent. The solids were redissolved in methyl t-butyl ether and analyzed by GC-MS and were found to contain principally methyl ester of cyclic glyoxylic acetal and 1,4-anhydroxylitol glyoxylate acetal, methyl ester (MeAXGA) as a 1.3:1 mixture of trans:cis stereoisomers.

The resulting polyacetal-polyester condensation product of 1,4-anhydroxylitol and glyoxylic acid was thus found to comprise cyclic acetal fragments of 1,4-anhydroxylitol and esterified glyoxylic acid, as well as undefined acyclic acetals which upon treatment with methanolic sodium ethoxide produced compounds insoluble in methyl t-butyl ether.

### Example 22

A 1 L beaker was charged with 160 g ethyl acetate and 200 g EtAXLK prepared according to the procedure of Example 2. The mixture was heated to 60° C. with magnetic stirring until EtAXLK was completed dissolved in ethyl acetate. Then the mixture was slowly cooled to −20° C. After about 2 hours at −20° C., a substantial amount of crystalline appearing precipitate (approximately 70 g) formed in the beaker. The precipitate was isolated by filtration, washed twice with cold (−20° C.) ethyl acetate and dried in a vacuum oven at 40° C. at 15 Torr for 3 days before subjecting to GC-MS analysis. The GC-MS data showed that the precipitate was 100% EtAXLK and the trans:cis isomer ratio was greater than 300:1.

### Example 23

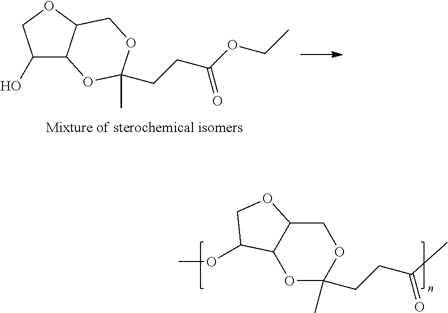

A 250 mL 3-neck flask equipped with a mechanical stirrer and a Dean-Stark trap with condenser and nitrogen/vacuum inlet was charged with 30.70 g (0.118 mol) EtAXLK obtained using the procedure of Example 2. The flask was evacuated to 200 mTorr and heated in an 85° C. oil bath with stirring. After 4 cycles of evacuating the flask to 200 mTorr and back-filling the flask with nitrogen, the flask was placed under vacuum at <100 mTorr and stirred in an 85° C. oil bath overnight. Then the flask was backfilled and under nitrogen blanket, the flask was charged with 6 μL titanium (IV) isopropoxide. After 3 cycles of evacuating to 200 mTorr and back-filling with nitrogen, the flask was heated to 200° C. in an oil bath under nitrogen atmosphere and with stirring. Over the next 7.5 hours, 4.0 mL of a liquid was collected in the Dean-Stark trap. The liquid was drained from the trap, and a vacuum of about 16-2.6 Torr was applied to the reaction flask for about one hour. Then high vacuum (about 100 mTorr) was applied and maintained overnight with an oil bath temperature set to 120° C. without stirring. Then the oil bath temperature was raised to 210° C., and mechanical stirring was resumed for about 1.5 hours, then the oil bath temperature was raised to 220° C. and stirring was continued for about 1.5 h, then finally the oil bath temperature was raised to 230° C., pressure was decreased to about 55-70 mTorr, and stirring was continued for about another 10 hours. Then the flask was cooled to 160° C. and backfilled with nitrogen.

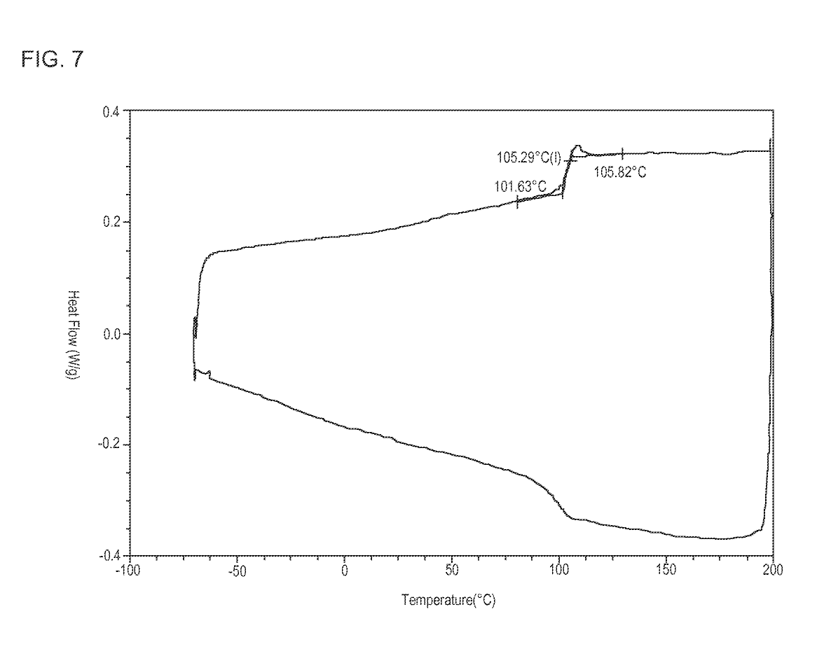

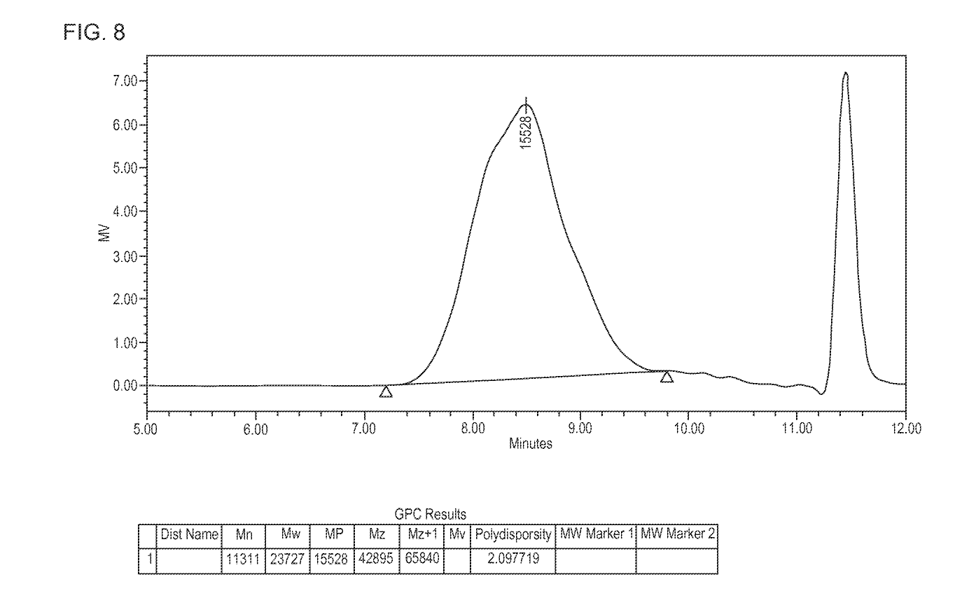

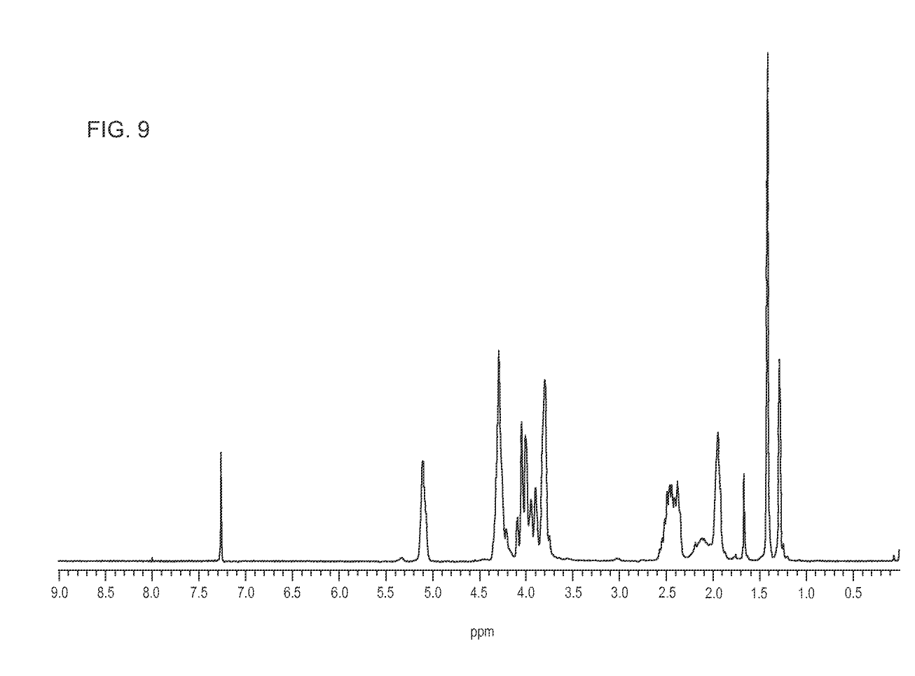

The reaction product was collected under nitrogen at 160° C. as a light yellow transparent solid. Yield was 17.45 g. The polymer was analyzed by DSC, GPC, and ¹H NMR. DSC (−70° to 200° C., 10° C./min) showed T_{g}=105.3° C.; the DSC trace is shown in FIG. 7. GPC (THF mobile phase, PS calibration, high MW column) showed Mₙ=11,300, PDI=2.10; the GPC trace is shown in FIG. 8. The ¹H NMR spectrum is shown in FIG. 9.

### Example 24

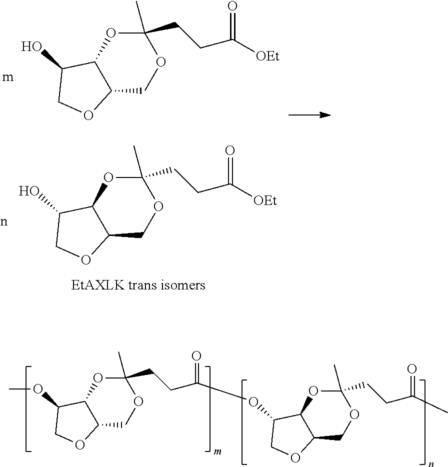

A 500 ml 3-neck round bottom flask was charged with 50 g (0.19 mol) of EtAXLK prepared according to the Example 23. The flask was equipped with a mechanical stirrer, a Dean-Stark trap with overhead condenser, and vacuum/nitrogen inlet. The flask was immersed in an oil bath set to a temperature of 110° C. and five vacuum/nitrogen degassing cycles were carried out at the elevated temperature. The flask was backfilled with nitrogen after degassing, and 10 μl (200 ppm) of titanium (IV) isoproxide were added to the reaction flask. Then, the temperature of the oil bath was increased to about 200°-210° C. and the contents of the flask were stirred for approximately 4 hours, during which time liquid was observed to collect in the Dean Stark trap. At the end of 4 hours the collection of liquid in the Dean Stark trap has subsided. Then, a vacuum of about 17 Torr was applied to the flask for approximately 4 hours while the temperature of the oil bath was maintained at 220° C. Then the oil bath temperature was increased to 240° C. and a vacuum of about 0.2 Torr was applied to the flask for additional 7 hours. Then the contents of the flask were allowed to cool to room temperature (about 20° C.), and the flask was backfilled with nitrogen.

The polymer product was retrieved and weighed about 36 g. A sample of the contents of was analyzed by GPC, which showed a weight average molecular weight (M_{w}) of 75,000 g/mol and number average molecular weight (Mₙ) of 22,000 g/mol. DSC analysis of the product was carried out according to the procedure of Example 23, and showed T_{g} of 115° C. The polymer was transparent, rigid, and ductile with good optical clarity.

### Example 25

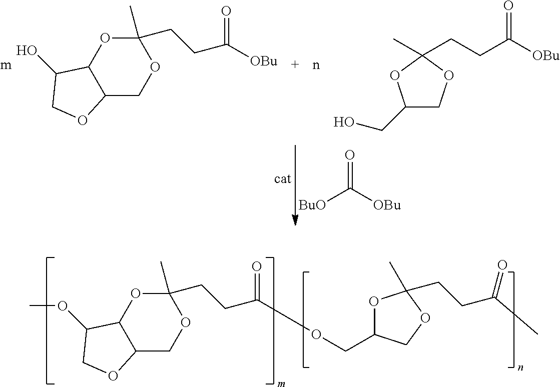

Di-n-butyl carbonate was prepared as follows. A screw cap bottle was charged with 2.1 mol of diethyl carbonate and 6.03 mol of n-butanol, then 0.2 g of sodium methoxide was added. The resulting mixture was magnetically stirred at room temperature (about 22° C.) for 8 hours. GC-MS analysis indicated that the resulting crude reaction product was 98% di-n-butyl carbonate, 2% ethyl n-butyl carbonate. Di-n-butyl carbonate was subjected to fractional distillation at about 4 Torr, wherein the major fraction was collected as colorless liquid at 72-76° C. and resulted in a 64 mol % yield based on starting amount of diethyl carbonate.

A 250 ml 3-neck round bottom flask was charged with 24.7 g (0.086 mol) BuAXLK prepared according to the procedure of Example 17, 0.75 mL (0.0043 mol) of di-n-butyl carbonate and 11.2 g (0.046 mol) of glycerol levulinate ketal, butyl ester (prepared according the procedure of Patent Application No. WO 2009/048874, Example 2, except that butyl levulinate was used instead of ethyl levulinate). The flask was equipped with a mechanical stirrer, a Dean-Stark trap with overhead condenser, vacuum/nitrogen inlet, and a thermocouple, and immersed in an oil bath set to a temperature of 60° C. Five vacuum/nitrogen degassing cycles were carried out, then the flask was backfilled with nitrogen and 14 μl (400 ppm) of titanium isoproxide (obtained from Thermo Fisher Scientific of Waltham, Mass.) were added to the flask. Then, the temperature of the contents of the flask was increased to about 200°-220° C. and the flask was stirred for approximately 5 hours, during which time a liquid was observed to collect in the Dean Stark trap. At the end of 5 hours the condensation of liquid was observed to stop. Then, a vacuum of about 7 Torr was applied to the flask for approximately 5 hours while the temperature was maintained at about 220° C. Finally, the temperature of the contents of the flask was raised to about 240° C. and a vacuum of about 0.2 Torr was applied, and stirring was continued for an additional 10 hours. The contents of the flask were then allowed to cool to room temperature.

A sample of the contents of the reaction flask was removed for GPC analysis according to the procedure of Example 23, which showed a weight average molecular weight (M_{w}) of 18,326 g/mol and number average molecular weight (Mₙ) of 6,597 g/mol. DSC was carried out according to the procedure of Example 23, which showed T_{g} of 66° C.

### Example 26

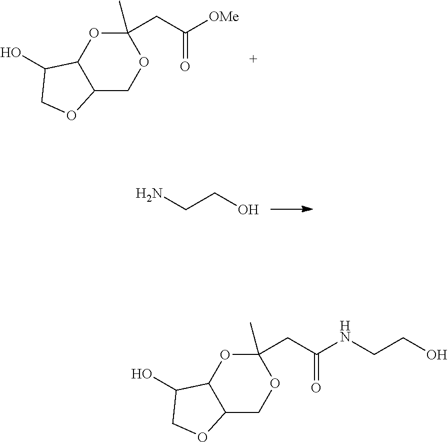

A 250 ml 3-neck round bottom flask was charged with 20.3 g (0.088 mol) of MeAXAK (prepared according to the procedure of Example 19), 7.2 g (0.12 mol) of ethanolamine and 6.0 mg (0.043 mmol) of 1,5,7-triazabicyclo[4.4.0]dec-5-ene. The flask was equipped with an overhead mechanical stirrer, a Dean-Stark trap with overhead condenser, and a vacuum/nitrogen inlet, and was immersed in an oil bath set to 160°-180° C. The contents of the flask were blanketed with a nitrogen stream and stirred for about 4 hours. During this time, a distillate was collected in the Dean Stark trap. Then the flask was evacuated to a pressure of about 10 Torr to remove excess ethanolamine by distillation. The resulting reaction product was analyzed by GC-MS to identify the reaction product as the 1,4-anhydroxylitol acetoacetamide ketal, 2-hydroxyethyl amide (mixture of stereoisomers). The analysis showed that no residual MeAXAK was present in the flask.

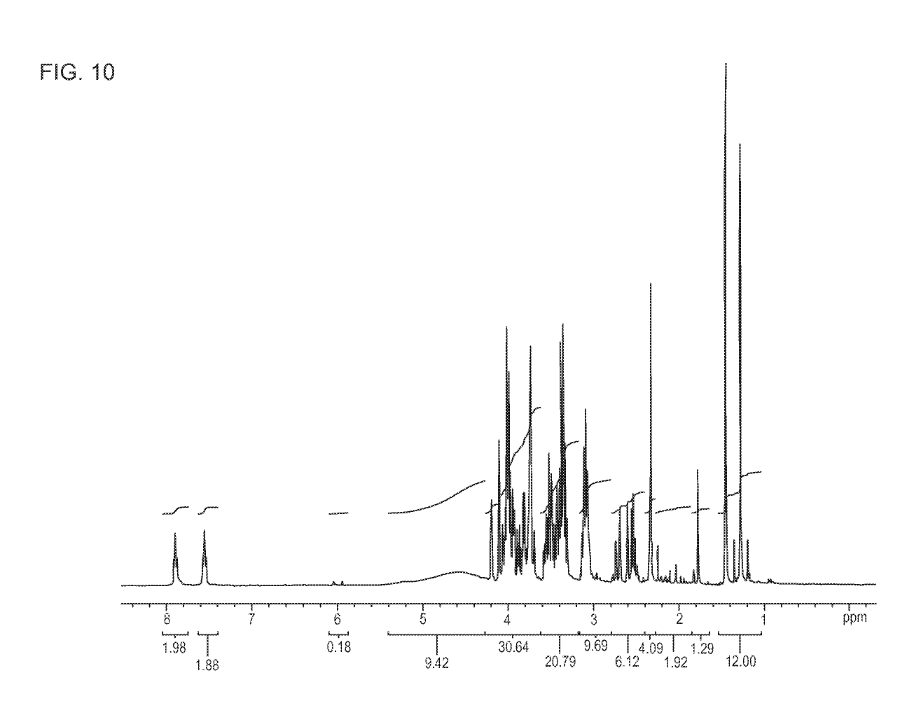

A sample of the reaction mixture was removed for ¹H NMR analysis, shown in FIG. 10.

### Example 27

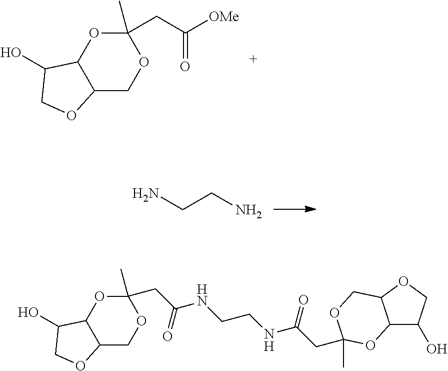

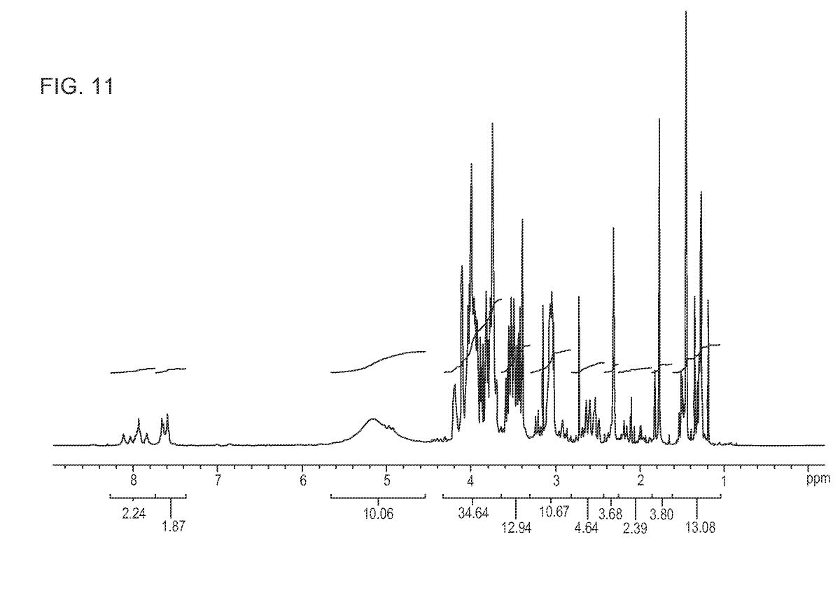

A 250 ml 3-neck round bottom flask was charged with 15.4 g (0.066 mol) of MeAXAK (prepared according to the Example 19), 1.96 g (0.033 mol) of ethylenediamine, and 5.0 mg (0.036 mmol) of 1,5,7-triazabicyclo[4.4.0]dec-5-ene (TBD). The flask was equipped with an overhead mechanical stirrer, a Dean-Stark trap with overhead condenser, and vacuum/nitrogen inlet and immersed in an oil bath set to 120° C. The contents of the flask were blanketed with a nitrogen stream and stirred for approximately 20 hours. During this time, distillates were collected in the Dean Stark trap. Then the temperature of the bath was slowly raised to 160° C. over a period of about 5 hours and held at 160° C. for another 13 hours. After the 13 hours, no further distillates were collected in the Dean Stark trap. The flask was allowed to cool to room temperature. Upon cooling, the resulting reaction product (about 14 g) was obtained as a transparent amber-colored solid. A sample of the reaction mixture was removed for ¹H NMR analysis, as shown in FIG. 11.

### Example 28

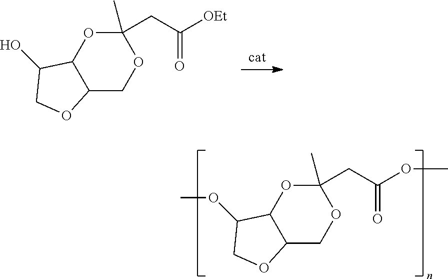

A 250 mL 3-neck round bottom flask was charged with 35 g (0.14 mol) EtAXAK prepared according to the procedure of Example 18. The flask was equipped with a mechanical stirrer, Dean-Stark trap with overhead condenser, and vacuum/nitrogen inlet. The flask was immersed in an oil bath set to a temperature of about 70° C., and 10 vacuum/nitrogen degassing cycles were carried out. The flask was then backfilled with nitrogen after degassing, and 14 mg (400 ppm) of 1,5,7-triazabicyclo[4.4.0]dec-5-ene were added to the reaction flask. The oil bath temperature was increased to 180°-200° C. and the contents of the flask were stirred for 4 hours, during which time liquid was observed to collect in the Dean Stark trap. At the end of 4 hours the distillation subsided. Then the oil bath temperature was increased to 200°-210° C. and pressure in the flask was reduced to about 22 Torr for about 2 hours, then pressure was reduced again to about 0.2 Torr for an additional 4 hours. Then the contents of the flask were allowed to cool to room temperature (about 21° C.), and the flask was backfilled with nitrogen.

The contents of the flask were removed and analyzed by GPC according to the procedure of Example 23, which showed the reaction product had a weight average molecular weight (M_{w}) of 2992 g/mol and number average molecular weight (Mₙ) of 2206 g/mol.

### Example 29

A 20 ml glass vial was charged with 1.84 g (7 mmol) of the bisamide reaction product of Example 27, and 15 ml of anhydrous dimethyl sulfoxide was added to completely dissolve the compound. Then, 1.6 g (7 mmol) of isophorone diisocyanate was added to the vial. The reaction mixture was stirred by means of magnetic stirring at ambient temperature (about 22° C.) for about 2 hours. Then 5 ml of methanol was added to the vial, and the reaction mixture was stirred for an additional 30 minutes. A sample of the reaction mixture was removed for GPC analysis according to the procedure of Example 23, which showed a compound having a weight average molecular weight (M_{w}) of 1784 g/mol and number average molecular weight (Mₙ) of 1624 g/mol.

The present invention may suitably comprise, consist of, or consist essentially of, any of the disclosed or recited elements and embodiments. A listing of some embodiments of the invention now follows. The invention illustratively disclosed herein can be suitably practiced in the absence of any element which is not specifically disclosed herein. The various embodiments described are provided by way of illustration only and should not be construed to limit the claims attached hereto. It will be recognized that various modifications and changes may be made without following the example embodiments and applications illustrated and described herein, and without departing from the true spirit and scope of the claims to follow.

A first embodiment of the invention, either alone or in combination with any other embodiment or combination of embodiments listed herein, contemplates a compound comprising one cyclic ketal group, one ester or amide group, and one cyclic ether group, wherein the compound is the product of the reaction of an anhydropentitol and a levulinate ester, or an anhydropentitol and a ketal of levulinate ester, or the ketal of an anhydropentitol and a levulinate ester. In any such first embodiment, either alone or in combination with any other embodiment or combination of embodiments listed herein, the reaction is a condensation reaction. In any such first embodiment, either alone or in combination with any other embodiment or combination of embodiments listed herein, the reaction is an exchange reaction. In any such first embodiment, either alone or in combination with any other embodiment or combination of embodiments listed herein, the anhydropentitol comprises 1,4-anhydroxylitol or 1,4-anhydroarabitol. In any such first embodiment, either alone or in combination with any other embodiment or combination of embodiments listed herein, the reaction is an exchange reaction of the methyl isobutyl ketal of 1,4-anhydropentitol with ethyl levulinate. In any such first embodiment, either alone or in combination with any other embodiment or combination of embodiments listed herein, the compound further comprises the residue of a fatty acid, a diacid, a diol, a diamine, an aminoalcohol, a ketal ester, glycerol carbonate, or a combination thereof. In any such first embodiment, either alone or in combination with any other embodiment or combination of embodiments listed herein, the residue of the ketal ester has the structure

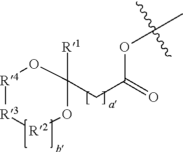

wherein
a′ is 0, 1, or 2;
b′ is 0 or 1, such that b=0 indicates a 5-membered ring and b=1 indicates a 6-membered ring;
R′¹ is hydrogen or methyl; and
R′², R′³, and R′⁴ are independently methylene, alkylmethylene, or dialkylmethylene.

In any such first embodiment, either alone or in combination with any other embodiment or combination of embodiments listed herein, the ketal ester is the ketal of an alkyl levulinate and 1,2-ethanediol, 1,2-propanediol, or a mixture thereof.

A second embodiment of the invention, either alone or in combination with any other embodiment or combination of embodiments listed herein, contemplates a formulation comprising one or more compounds of the first embodiment described above and one or more polymers, surfactants, plasticizers, solvents, colorants, catalysts, fillers, additives, adjuvants, or a combination thereof. In any such second embodiment, either alone or in combination with any other embodiment or combination of embodiments listed herein, the formulation is a plasticized polymer formulation, a coating formulation, an ink formulation, an adhesive formulation, a cleaning formulation, or a personal care formulation.

A third embodiment of the invention, either alone or in combination with any other embodiment or combination of embodiments listed herein, contemplates an article comprising one or more formulations of the second embodiment. In any such third embodiment, either alone or in combination with any other embodiment or combination of embodiments listed herein, the article is a film, a fiber, an extrusion molded article, an injection molded article, a cast article, a foamed article, or a coating.

A fourth embodiment of the invention, either alone or in combination with other embodiments listed herein, contemplates a polymer comprising at least one repeat unit comprising the residue of a compound comprising one cyclic ketal group, one ester or amide group, and one cyclic ether group, wherein the compound is the product of the reaction of an anhydropentitol and a levulinate ester, or an anhydropentitol and a ketal of levulinate ester, or the ketal of an anhydropentitol and a levulinate ester. In any such fourth embodiment, either alone or in combination with any other embodiments or combination of embodiments listed herein, the polymer comprises the residue of one or more compounds of the first embodiment. In any such fourth embodiment, either alone or in combination with any other embodiments or combination of embodiments listed herein, the polymer further comprises the residue of a diacid, a diol, a diamine, an aminoalcohol, a diisocyanate, a hydroxyacid or hydroxyester, hydroxyketal ester, or a combination thereof. In any such fourth embodiment, either alone or in combination with any other embodiments or combination of embodiments listed herein, the hydroxyketal ester comprises the structure

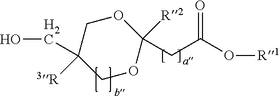

wherein
a″ is 0, 1, or 2,
b″ is 0 or 1, and
R″¹, R″², and R″³ are independently hydrogen or an alkyl group having between 1 and 6 carbon atoms.

In any such fourth embodiment, either alone or in combination with any other embodiments or combination of embodiments listed herein, a″ is 2, b″ is 0, R″² is methyl, and R″³ is hydrogen. In any such fourth embodiment, either alone or in combination with any other embodiments or combination of embodiments listed herein, the polymer comprises between 1 and 500 repeat units comprising the residue of one or more compounds of the first embodiment. In any such fourth embodiment, either alone or in combination with any other embodiments or combination of embodiments listed herein, the glass transition temperature of the polymer is between 50° C. and 150° C. In any such fourth embodiment, either alone or in combination with any other embodiments or combination of embodiments listed herein, the glass transition temperature of the polymer is between 100° C. and 150° C. In any such fourth embodiment, either alone or in combination with any other embodiments or combination of embodiments listed herein, the polymer is substantially transparent.

A fifth embodiment of the invention contemplates a formulation comprising one or more polymers of the fourth embodiment and one or more additional polymers, crosslinkers, surfactants, solvents, colorants, fillers, plasticizers, tackifiers, catalysts, additives, impact modifiers, adjuvants, UV stabilizers, thermal stabilizers, antimicrobial agents, antifungal agents, antiviral agents, bleaches, or a combination thereof. In any such fifth embodiment, either alone or in combination with any other embodiments or combination of embodiments listed herein, the formulation is a cleaning formulation, a degreasing formulation, an adhesive formulation, a coating formulation, an ink, or a personal care formulation. In any such fifth embodiment, either alone or in combination with any other embodiments or combination of embodiments listed herein, the formulation further comprises glycerol carbonate, a cyclic ketal of an alkyl levulinate with glycerol, erythritol, sorbitol, or anhydroxylitol, or a combination of one or more thereof.

A sixth embodiment of the invention, either alone or in combination with any other embodiments or combination of embodiments listed herein, contemplates an article comprising one or more polymers of the fourth embodiment, wherein the article is a film, a fiber, an extrusion molded item, a cast item, an extrusion formed article, an injection molded article, a skived article, a foamed article, or a coating. In any such sixth embodiment, either alone or in combination with any other embodiments or combination of embodiments listed herein, the article is a comestibles container such as a cup, plate, bottle, or punnet, or a utensil for use with comestibles such as a fork, spoon, or knife; a film for protecting food, a video screen, a window, a window or door component; a flexible cable; an insulating film for magnet wire; a medical article requiring sterilization, such as a tube or a bag or a component thereof; a photoresist component; a structural adhesive component; a bushing, bearing, or socket; a component of a hot gas filter; a data storage device such as a compact disc or component of a data storage device such as a solid state drive or flash memory device; a casing or housing for a computer, printer, MP3 player, cellular phone, camera, video display device, stereo speaker, power strip, or electrical connector; an item of furniture or a component thereof such as a table top, lamp base, desk, or chair; a lense, cover, or diffuser for lighting, such as a streetlight lamp cover, an eyeglass or sunglass lense, a diffuser for a fluorescent bulb; an automotive part such as a steering wheel, an instrument panel component or cover, an interior molded part such as a dashboard component, or an exterior molded part such as an automotive headlamp or a bumper; a component of protective gear such as a shield, a helmet, or a visor; a children's toy such as a spinning top or a remote control car body; or a protective cover for a poster, photograph, billboard, compact disc, book, or notebook. In any such sixth embodiment, either alone or in combination with any other embodiments or combination of embodiments listed herein, the article of further comprises one or more polymers, crosslinkers, surfactants, solvents, colorants, fillers, plasticizers, tackifiers, catalysts, additives, impact modifiers, adjuvants, UV stabilizers, thermal stabilizers, antimicrobial agents, antifungal agents, antiviral agents, bleaches, or a combination thereof.

A seventh embodiment of the invention, either alone or in combination with any other embodiments or combination of embodiments listed herein, contemplates a compound comprising a structure I,

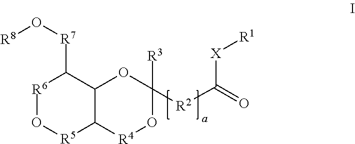

wherein
a is 0 or an integer of 1 to 12;

X is O or NR, wherein R is hydrogen or a linear or branched alkyl group having between 1 and 6 carbons;
R¹ is hydrogen, a metal cation, an organic cation, a linear, branched, or cyclic alkyl, a linear, branched, or cyclic alkenyl, alkynyl, aryl, alkaryl, or an oligomeric or polymeric moiety comprising ethylene oxide, propylene oxide, or a combination thereof;
each R² is methylene, alkylmethylene, or dialkylmethylene;
R³ is hydrogen, an alkynyl group, or a linear, branched, or cyclic alkyl or alkenyl group having 1 to 18 carbon atoms, or an aryl or alkaryl group;
one of R⁴ and R⁵ is a methylene, alkylmethylene, or dialkylmethylene group and the other is a covalent bond;
one of R⁶ and R⁷ is a methylene, alkylmethylene, or dialkylmethylene group and the other is a covalent bond, with the proviso that R⁵ and R⁶ are not simultaneously a covalent bond; and
R⁸ is hydrogen or an acyl group having a linear, branched, or cyclic alkyl or alkenyl group, aryl group, or aralkyl group.

In any such seventh embodiment of the invention, either alone or in combination with any other embodiments or combination of embodiments listed herein, one or more of R¹, R², R³, R⁷, and R⁸ further comprise one or more heteroatoms comprising oxygen, nitrogen, sulfur, silicon, phosphorus, or a halogen. In any such seventh embodiment of the invention, either alone or in combination with any other embodiments or combination of embodiments listed herein, the compound is a mixture of isomers such that some R⁴ are methylene, alkylmethylene, or dialkylmethylene groups, some R⁶ are methylene, alkylmethylene, or dialkylmethylene groups, and some R⁷ are methylene, alkylmethylene, or dialkylmethylene groups. In any such seventh embodiment of the invention, either alone or in combination with any other embodiments or combination of embodiments listed herein, the mixture of isomers comprises from 50 to 100 mol % of the isomer wherein R⁶ and R⁷ are covalent bonds. In any such seventh embodiment of the invention, either alone or in combination with any other embodiments or combination of embodiments listed herein, the mixture of isomers comprises from 95 to 100 mol % of the isomer wherein R⁶ and R⁷ are covalent bonds. In any such seventh embodiment of the invention, either alone or in combination with any other embodiments or combination of embodiments listed herein, R⁶ and R⁷ are covalent bonds. In any such seventh embodiment of the invention, either alone or in combination with any other embodiments or combination of embodiments listed herein, R⁴ is methylene. In any such seventh embodiment of the invention, either alone or in combination with any other embodiments or combination of embodiments listed herein, the compound comprises the residue of anhydroxylitol. In any such seventh embodiment of the invention, either alone or in combination with any other embodiments or combination of embodiments listed herein, a is 2, all R² are methylene, and R³ is methyl. In any such seventh embodiment of the invention, either alone or in combination with any other embodiments or combination of embodiments listed herein, X is O and R¹ is selected from methyl, ethyl, isopropyl, and n-butyl. In any such seventh embodiment of the invention, either alone or in combination with any other embodiments or combination of embodiments listed herein, X is NR and R¹ comprises a residue having the structure

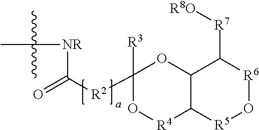

wherein R, R², R³, R⁴, R⁵, R⁶, R⁷, R⁸, and a are independently as defined as for compound I. In any such seventh embodiment of the invention, either alone or in combination with any other embodiments or combination of embodiments listed herein, R⁸ is hydrogen, a fatty acid ester residue, or a benzoyl group. In any such seventh embodiment of the invention, either alone or in combination with any other embodiments or combination of embodiments listed herein, R⁸ is a ketal ester residue having the structure

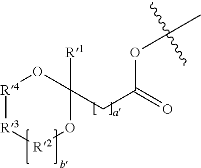

wherein
a′ is 0, 1, or 2;
b′ is 0 or 1, such that b=0 indicates a 5-membered ring and b=1 indicates a 6-membered ring;
R′¹ is hydrogen or methyl; and
R′², R′³, and R′⁴ are independently methylene, alkylmethylene, or dialkylmethylene.

In any such seventh embodiment of the invention, either alone or in combination with any other embodiments or combination of embodiments listed herein, the ketal ester comprises an alkyl levulinate and 1,2-ethanediol or 1,2-propanediol. In any such seventh embodiment of the invention, either alone or in combination with any other embodiments or combination of embodiments listed herein, R¹ is the residue of glycerol carbonate. In any such seventh embodiment of the invention, either alone or in combination with any other embodiments or combination of embodiments listed herein, the compound comprises a mixture of isomers comprising less than 50 mol % cis isomers and more than 50 mol % trans isomers, wherein the cis isomers are

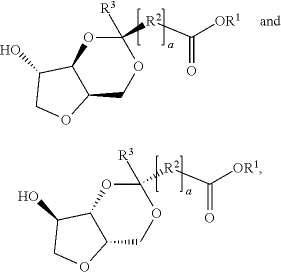

and the trans isomers are

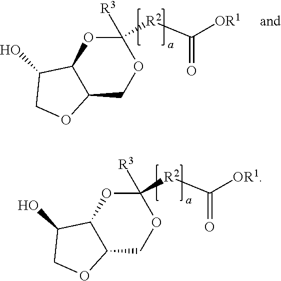

In any such seventh embodiment of the invention, either alone or in combination with any other embodiments or combination of embodiments listed herein, all a are 2, all R³ are methyl, all R² are methylene, and all R¹ are ethyl or n-butyl. In any such seventh embodiment of the invention, either alone or in combination with any other embodiments or combination of embodiments listed herein, the molar ratio of trans:cis is between 2:1 and 500:1. In any such seventh embodiment of the invention, either alone or in combination with any other embodiments or combination of embodiments listed herein, the molar ratio of trans:cis is between 5:1 and 100:1. In any such seventh embodiment of the invention, either alone or in combination with any other embodiments or combination of embodiments listed herein, the compound consists essentially of trans isomers.

An eighth embodiment of the invention, either alone or in combination with any other embodiments or combination of embodiments listed herein, contemplates a formulation comprising a compound of the seventh embodiment of the invention and one or more polymers, crosslinkers, surfactants, solvents, catalysts, colorants, fillers, additives, adjuvants, or a combination thereof. In any such eighth embodiment of the invention, either alone or in combination with any other embodiments or combination of embodiments listed herein, the polymer is polyvinyl chloride or an ethylene-vinyl acetate copolymer. In any such eighth embodiment of the invention, either alone or in combination with any other embodiments or combination of embodiments listed herein, the formulation is a cleaning formulation, a degreasing formulation, an adhesive formulation, a coating formulation, an ink, or a personal care formulation.

A ninth embodiment of the invention, either alone or in combination with any other embodiments or combination of embodiments listed herein, contemplates an article comprising one or more formulations of the eighth embodiment, wherein the article is a film, a fiber, an extrusion molded item, an injection molded article, a cast article, a foamed article, or a coating.

A tenth embodiment of the invention, either alone or in combination with any other embodiments or combination of embodiments listed herein, contemplates a polymer comprising at least one repeat unit comprising structure II:

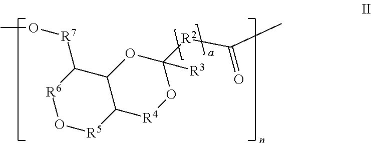

wherein
a is 0 or an integer of 1 to 12;
each R² is methylene, alkylmethylene, or dialkylmethylene;
R³ is hydrogen, an alkynyl group, or a linear, branched, or cyclic alkyl or alkenyl group having 1 to 18 carbon atoms, or an aryl or alkaryl group;
one of R⁴ and R⁵ is a methylene, alkylmethylene, or dialkylmethylene group and the other is a covalent bond;
one of R⁶ and R⁷ is a methylene, alkylmethylene, or dialkylmethylene group and the other is a covalent bond, with the proviso that R⁵ and R⁶ are not simultaneously a covalent bond, and
n is an integer of between 1 and 500.

In any such tenth embodiment of the invention, either alone or in combination with any other embodiments or combination of embodiments listed herein, the glass transition temperature of the polymer is between about 50° C. and 150° C. In any such tenth embodiment of the invention, either alone or in combination with any other embodiments or combination of embodiments listed herein, the polymer is a homopolymer and the glass transition temperature is between about 80° C. and 150° C. In any such tenth embodiment of the invention, either alone or in combination with any other embodiments or combination of embodiments listed herein, one or more of R², R³, and R⁷ further comprise one or more heteroatoms comprising oxygen, nitrogen, sulfur, silicon, phosphorus, or a halogen. In any such tenth embodiment of the invention, either alone or in combination with any other embodiments or combination of embodiments listed herein, the polymer further comprises the residue of a diacid, a diol, a diamine, an aminoalcohol, a diisocyanate, a hydroxyacid, or hydroxyester, hydroxyketal ester, or a combination thereof. In any such tenth embodiment of the invention, either alone or in combination with any other embodiments or combination of embodiments listed herein, the hydroxyketal ester comprises the structure

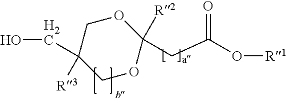

wherein
a″ is 0, 1, or 2,
b″ is 0 or 1 such that b=0 indicates a 5-membered ring and b=1 indicates a 6-membered ring, and
R″¹, R″₂, and R″₃ are independently hydrogen or an alkyl group having between 1 and 6 carbon atoms.

In any such tenth embodiment of the invention, either alone or in combination with any other embodiments or combination of embodiments listed herein, a″ is 2, b″ is 0, R″² is methyl, and R″³ is hydrogen. In any such tenth embodiment of the invention, either alone or in combination with any other embodiments or combination of embodiments listed herein, the polymer further comprises urethane, urea, acrylate, epoxy, carbamate, or carbonate functionality, or a combination thereof. In any such tenth embodiment of the invention, either alone or in combination with any other embodiments or combination of embodiments listed herein, the polymer comprises one or more repeat units wherein R⁴ is a methylene, alkylmethylene, or dialkylmethylene group, one or more repeat units wherein R⁶ is a methylene, alkylmethylene, or dialkylmethylene group, and one or more repeat units wherein R⁷ is a methylene, alkylmethylene, or dialkylmethylene group. In any such tenth embodiment of the invention, either alone or in combination with any other embodiments or combination of embodiments listed herein, the one or more repeat units wherein R⁴ is a methylene, alkylmethylene, or dialkylmethylene group comprises from 50 to 100 mol % of the total number of repeat units comprising structure II. In any such tenth embodiment of the invention, either alone or in combination with any other embodiments or combination of embodiments listed herein, R⁶ and R⁷ are covalent bonds. In any such tenth embodiment of the invention, either alone or in combination with any other embodiments or combination of embodiments listed herein, R⁴ and R⁵ are methylene. In any such tenth embodiment of the invention, either alone or in combination with any other embodiments or combination of embodiments listed herein, one or more repeat units of the polymer comprises the residue of anhydroxylitol. In any such tenth embodiment of the invention, either alone or in combination with any other embodiments or combination of embodiments listed herein, all a is 2, all R² are methylene, and R³ is methyl. In any such tenth embodiment of the invention, either alone or in combination with any other embodiments or combination of embodiments listed herein, the polymer further comprises one or more endgroups comprising hydrogen, an alkoxy group, or the residue of an alkyl ester, a fatty acid ester, glycerol carbonate, a ketal ester, a diol, or a diester. In any such tenth embodiment of the invention, either alone or in combination with any other embodiments or combination of embodiments listed herein, the ketal ester residue has the structure

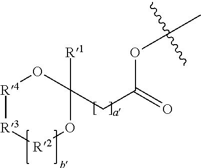

wherein
a′ is 0, 1, or 2;
b′ is 0 or 1, such that b=0 indicates a 5-membered ring and b=1 indicates a 6-membered ring;
R′¹ is hydrogen or methyl; and
R′², R′³, and R′⁴ are independently methylene, alkylmethylene, or dialkylmethylene.

In any such tenth embodiment of the invention, either alone or in combination with any other embodiments or combination of embodiments listed herein, the ketal ester is the ketal of an alkyl levulinate and 1,2-ethanediol or 1,2-propanediol. In any such tenth embodiment of the invention, either alone or in combination with any other embodiments or combination of embodiments listed herein, n is between 10 and 250. In any such tenth embodiment of the invention, either alone or in combination with any other embodiments or combination of embodiments listed herein, n is between 1 and 10. In any such tenth embodiment of the invention, either alone or in combination with any other embodiments or combination of embodiments listed herein, n is between 1 and 5. In any such tenth embodiment of the invention, either alone or in combination with any other embodiments or combination of embodiments listed herein, the polymer comprises a plurality of repeat units comprising the residue of 1,4-anhydroxylitol comprising less than 50 mol % cis isomers and more than 50 mol % trans isomers, wherein the cis isomers are

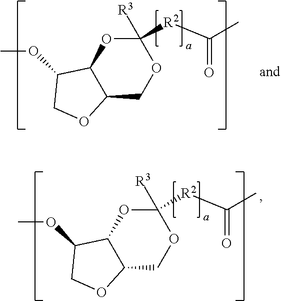

and the trans isomers are

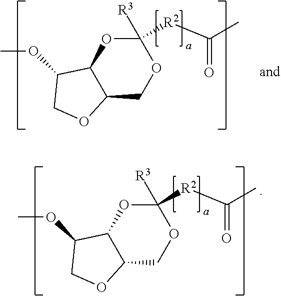

In any such tenth embodiment of the invention, either alone or in combination with any other embodiments or combination of embodiments listed herein, the ratio of trans:cis isomers is between 2:1 and 500:1. In any such tenth embodiment of the invention, either alone or in combination with any other embodiments or combination of embodiments listed herein, all a are 2, all R³ are methyl, and all R² are methylene. In any such tenth embodiment of the invention, either alone or in combination with any other embodiments or combination of embodiments listed herein, the compound is a homopolymer consisting essentially of trans isomers. In any such tenth embodiment of the invention, either alone or in combination with any other embodiments or combination of embodiments listed herein, the polymer is a copolymer. In any such tenth embodiment of the invention, either alone or in combination with any other embodiments or combination of embodiments listed herein, the glass transition temperature of the polymer is between 90° C. and 150° C. In any such tenth embodiment of the invention, either alone or in combination with any other embodiments or combination of embodiments listed herein, the glass transition temperature of compound is between 100° C. and 150° C. In any such tenth embodiment of the invention, either alone or in combination with any other embodiments or combination of embodiments listed herein, the glass transition temperature of the polymer is between about 110° C. and 150° C.

An eleventh embodiment of the invention, either alone or in combination with any other embodiments or combination of embodiments listed herein, contemplates a formulation comprising one or more polymers of the tenth embodiment and one or more polymers, crosslinkers, surfactants, solvents, colorants, fillers, plasticizers, tackifiers, catalysts, additives, impact modifiers, adjuvants, UV stabilizers, thermal stabilizers, antimicrobial agents, antifungal agents, antiviral agents, bleaches, or a combination thereof. In any such eleventh embodiment of the invention, either alone or in combination with any other embodiments or combination of embodiments listed herein, the formulation is a cleaning formulation, a degreasing formulation, an adhesive formulation, a coating formulation, an ink, or a personal care formulation. In any such eleventh embodiment of the invention, either alone or in combination with any other embodiments or combination of embodiments listed herein, the formulation further comprises glycerol carbonate, a cyclic ketal of an alkyl levulinate with glycerol, erythritol, sorbitol, or anhydroxylitol, or a combination of one or more thereof.

A twelfth embodiment of the invention, either alone or in combination with any other embodiments or combination of embodiments listed herein, contemplates an article comprising the polymer of the eleventh embodiment. In any such twelfth embodiment, either alone or in combination with any other embodiments or combination of embodiments listed herein, the article further comprises one or more polymers, crosslinkers, surfactants, solvents, colorants, fillers, plasticizers, tackifiers, catalysts, additives, impact modifiers, adjuvants, UV stabilizers, thermal stabilizers, antimicrobial agents, antifungal agents, antiviral agents, bleaches, or a combination thereof. In any such twelfth embodiment, either alone or in combination with any other embodiments or combination of embodiments listed herein, the article is a film, a fiber, an extrusion molded item, a cast item, an extrusion formed article, an injection molded article, a skived article, a foamed article, or a coating. In any such twelfth embodiment, either alone or in combination with any other embodiments or combination of embodiments listed herein, the article is a comestibles container such as a cup, plate, bottle, or punnet, or a utensil for use with comestibles such as a fork, spoon, or knife; a film for protecting food, a video screen, a window, a window or door component; a flexible cable; an insulating film for magnet wire; a medical article requiring sterilization, such as a tube or a bag or a component thereof; a photoresist component; a structural adhesive component; a bushing, bearing, or socket; a component of a hot gas filter; a data storage device such as a compact disc or component of a data storage device such as a solid state drive or flash memory device; a casing or housing for a computer, printer, MP3 player, cellular phone, camera, video display device, stereo speaker, power strip, or electrical connector; an item of furniture or a component thereof such as a table top, lamp base, desk, or chair; a lense, cover, or diffuser for lighting, such as a streetlight lamp cover, an eyeglass or sunglass lense, a diffuser for a fluorescent bulb; an automotive part such as a steering wheel, an instrument panel component or cover, an interior molded part such as a dashboard component, or an exterior molded part such as an automotive headlamp or a bumper; a component of protective gear such as a shield, a helmet, or a visor; a children's toy such as a spinning top or a remote control car body; or a protective cover for a poster, photograph, billboard, compact disc, book, or notebook.

A thirteenth embodiment of the invention, either alone or in combination with any other embodiments or combination of embodiments listed herein, contemplates a method of making a compound having structure I:

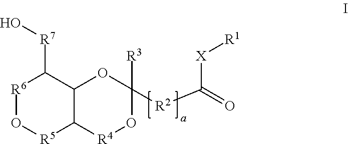

wherein
a is 0 or an integer of 1 to 12;
X is O or NR, wherein R is hydrogen or a linear or branched alkyl group having between 1 and 6 carbons;
R¹ is hydrogen, a metal cation, an organic cation, a linear, branched, or cyclic alkyl, a linear, branched, or cyclic alkenyl, alkynyl, aryl, alkaryl, or an oligomeric or polymeric moiety comprising ethylene oxide, propylene oxide, or a combination thereof;
each R² is methylene, alkylmethylene, or dialkylmethylene;
R³ is hydrogen, an alkynyl group, or a linear, branched, or cyclic alkyl or alkenyl group having 1 to 18 carbon atoms, or an aryl or alkaryl group;
one of R⁴ and R⁵ is a methylene, alkylmethylene, or dialkylmethylene group and the other is a covalent bond; and
one of R⁶ and R⁷ is a methylene, alkylmethylene, or dialkylmethylene group and the other is a covalent bond, with the proviso that R⁵ and R⁶ are not simultaneously a covalent bond,
   the method comprising
a. forming a cyclic anhydropentitol from a linear pentitol under conditions wherein water is removed; and
b. reacting the cyclic anhydropentitol with an oxocarboxylate to form the compound.

In any such thirteenth embodiment of the invention, either alone or in combination with any other embodiments or combination of embodiments listed herein, the method further comprises separating one or more stereoisomers of the compound. In any such thirteenth embodiment of the invention, either alone or in combination with any other embodiments or combination of embodiments listed herein, the method further comprises separating one or more stereoisomers of the compound by crystallization, recrystallization, or a combination thereof. In any such thirteenth embodiment of the invention, either alone or in combination with any other embodiments or combination of embodiments listed herein, the method further comprises adding a catalyst comprising sulfuric acid, sulfonic acid, a polymeric sulfonic acid, or a mixture thereof. In any such thirteenth embodiment of the invention, either alone or in combination with any other embodiments or combination of embodiments listed herein, the reacting comprises an exchange reaction. In any such thirteenth embodiment of the invention, either alone or in combination with any other embodiments or combination of embodiments listed herein, the exchange reaction comprises forming a ketal of the oxocarboxylate and a ketone, followed by exchange of the ketone with the anhydropentitol. In any such thirteenth embodiment of the invention, either alone or in combination with any other embodiments or combination of embodiments listed herein, the exchange reaction comprises forming a ketal of the anhydropentitol and a ketone, followed by exchange of the ketone with the oxocarboxylate. In any such thirteenth embodiment of the invention, either alone or in combination with any other embodiments or combination of embodiments listed herein, the anhydropentitol is anhydroxylitol and the oxocarboxylate is an alkyl levulinate. In any such thirteenth embodiment of the invention, either alone or in combination with any other embodiments or combination of embodiments listed herein, the anhydroxylitol comprises between about 50 and 100 mol % 1,4-anhydroxylitol and the ketone is 4-methyl-2-pentanone. In any such thirteenth embodiment of the invention, either alone or in combination with any other embodiments or combination of embodiments listed herein, the anhydroxylitol comprises between about 95 and 100 mol % 1,4-anhydroxylitol. In any such thirteenth embodiment of the invention, either alone or in combination with any other embodiments or combination of embodiments listed herein, the method is a continuous method.

We claim:

1. A polymer comprising at least one repeat unit having the structure: 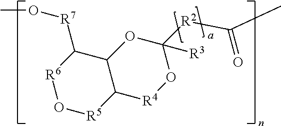
wherein:
a is 0 or an integer of 1 to 12;
each R² is independently methylene, alkylmethylene, or dialkylmethylene;
R³ is hydrogen, an alkynyl group, or a linear, branched, or cyclic alkyl or alkenyl group having 1 to 18 carbon atoms, or an aryl or alkaryl group;
one of R⁴, and R⁵, is a methylene, alkylmethylene, or dialkylmethylene group and the other is a covalent bond;
one of R⁶ and R⁷ is a methylene, alkylmethylene, or dialkylmethylene group and the other is a covalent bond, with the proviso that R⁵ and R⁶ are not simultaneously a covalent bond; and
n is an integer of between 1 and 500.

2. The polymer of claim 1 wherein all a are 2, all R² are methylene, and all R³ are methyl.

3. The polymer of claim 1 wherein the glass transition temperature of the polymer is between about 50° C. and 150° C.

4. The polymer of claim 1 wherein the polymer is a homopolymer and the glass transition temperature is between about 80° C. and 150° C.

5. The polymer of claim 1 wherein the polymer further comprises the residue of a diacid, a diol, a diamine, an aminoalcohol, a diisocyanate, a hydroxyacid, a hydroxyester, a hydroxyketal ester, or a combination thereof.

6. The polymer of claim 1 wherein the polymer further comprises the residue of a bislactamate.

7. The polymer of claim 6 wherein the bislactamate is carbonyl bis-N-caprolactamate.

8. The polymer of claim 1 wherein the compound comprises a plurality of repeat units comprising the residue of 1,4-anhydroxylitol, the repeat units comprising:

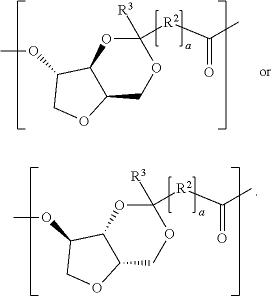

9. The polymer of claim 8 wherein all a are 2, all R² are methylene, and all R³ are methyl.

10. The polymer of claim 8 wherein the polymer is a homopolymer, wherein the homopolymer has a glass transition temperature in the range of about 75° C. to 130° C.

11. The polymer of claim 1 wherein the at least one repeat unit is derived from a compound having the structure 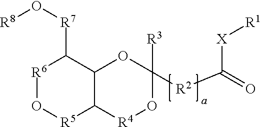
wherein:
a, R², R³, R⁴, R⁵, R⁶, and R⁷ are as defined in claim 1;
R¹ is hydrogen, a metal cation, an organic cation, a linear, branched, or cyclic alkyl, a linear, branched, or cyclic alkenyl, alkynyl, aryl, alkaryl, or an oligomeric or polymeric moiety comprising ethylene oxide, propylene oxide, or a combination thereof;
R⁸ is hydrogen or an acyl group having a linear, branched, or cyclic alkyl or alkenyl group, aryl group, or aralkyl group; and
X is O or NR, wherein R is hydrogen or a linear or branched alkyl group having between 1 and 6 carbons.

12. The polymer of claim 11 wherein the compound is a mixture of isomers comprising 50 to 100 mol % of the isomer wherein R⁶ and R⁷ are covalent bonds.

13. The polymer of claim 11 wherein the compound comprises a residue of 1,4-anhydroxylitol, the monomer compound comprising a mixture of isomers comprising:

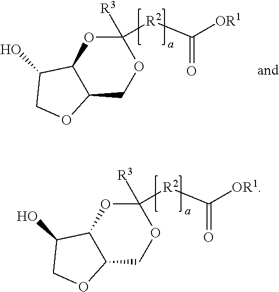

14. The polymer of claim 13 wherein all a are 2, all R² are methylene, and all R³ are methyl.

15. A formulation comprising
a. a polymer comprising at least one repeat unit having the structure: 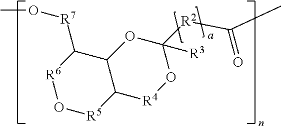
wherein:
a is 0 or an integer of 1 to 12,
each R² is independently methylene, alkylmethylene, or dialkylmethylene,
R³ is hydrogen, an alkynyl group, or a linear, branched, or cyclic alkyl or alkenyl group having 1 to 18 carbon atoms, or an aryl or alkaryl group,
one of R⁴, and R⁵, is a methylene, alkylmethylene, or dialkylmethylene group and the other is a covalent bond,
one of R⁶ and R⁷ is a methylene, alkylmethylene, or dialkylmethylene group and the other is a covalent bond, with the proviso that R⁵ and R⁶ are not simultaneously a covalent bond,
n is an integer of between 1 and 500; and
b. a crosslinker, surfactant, solvent, additional polymer, colorant, filler, plasticizer, tackifier, catalyst, additive, impact modifier, adjuvant, UV stabilizer, thermal stabilizer, antimicrobial agent, antifungal agent, antiviral agent, bleach, or a combination of one or more thereof.

16. The formulation of claim 15 wherein the formulation is a cleaning formulation, a degreasing formulation, an adhesive formulation, a coating formulation, an ink, or a personal care formulation.

17. The formulation of claim 15 wherein the formulation further comprises glycerol carbonate, a cyclic ketal of an alkyl levulinate with glycerol, erythritol, sorbitol, anhydroxylitol, or a combination of one or more thereof.

18. An article comprising a polymer comprising at least one repeat unit having the structure: 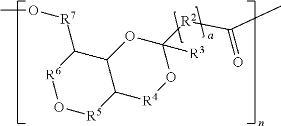
wherein:
a is 0 or an integer of 1 to 12,
each R² is independently methylene, alkylmethylene, or dialkylmethylene,
R³ is hydrogen, an alkynyl group, or a linear, branched, or cyclic alkyl or alkenyl group having 1 to 18 carbon atoms, or an aryl or alkaryl group,
one of R⁴, and R⁵, is a methylene, alkylmethylene, or dialkylmethylene group and the other is a covalent bond,
one of R⁶ and R⁷ is a methylene, alkylmethylene, or dialkylmethylene group and the other is a covalent bond, with the proviso that R⁵ and R⁶ are not simultaneously a covalent bond, and
n is an integer of between 1 and 500;
and wherein the article is a film, a fiber, an extrusion molded item, a compression molded item, a cast item, a foamed article, or a coating.

19. The article of claim 18 wherein the article further comprises one or more crosslinkers, additional polymers, surfactants, solvents, colorants, fillers, plasticizers, tackifiers, catalysts, additives, impact modifiers, adjuvants, UV stabilizers, thermal stabilizers, antimicrobial agents, antifungal agents, antiviral agents, bleaches, or a combination thereof.
